(19) United States
(12) Patent Application Publication
(10) Pub. No.: US 2021/0369725 A1
(43) Pub. Date: Dec. 2, 2021

(54) SELECTIVE TREATMENT OF CANCERS HAVING HISTONE H3 MUTATIONS OR ABERRANT LEVELS OF DNA OR HISTONE METHYLATION, ACETYLATION OR DEFECTS IN HOMOLOGOUS RECOMBINATION

(71) Applicant: FLORIDA STATE UNIVERSITY RESEARCH FOUNDATION, INC., TALLAHASSEE, FL (US)

(72) Inventor: AKASH GUNJAN, TALLAHASSEE, FL (US)

(21) Appl. No.: 17/303,457

(22) Filed: May 28, 2021

Related U.S. Application Data
(60) Provisional application No. 63/032,653, filed on May 31, 2020.

(51) Int. Cl. A61K 31/5377; A61K 31/404; A61K 31/4741; A61K 31/522; A61K 31/517; A61K 45/06

(52) U.S. Cl. CPC A61K 31/5377; A61K 31/404; A61K 45/06; A61K 31/522; A61K 31/517; A61K 31/4741

(57) ABSTRACT

The invention concerns compositions and a method for treating or delaying the onset, progression, or relapse of a cancer in a subject, the method comprising administering, optionally with radiation therapy, an inhibitor of: (a) DNA-Dependent Protein Kinase catalytic subunit, and an inhibitor of Poly-ADP Ribose Polymerase, wherein the cancer has a histone H3.3 mutation or is a homologous recombination-defective (HR-defective) cancer; or (b) wild-type isocitrate dehydrogenase, wherein the IDH inhibitor is administered in an effective amount to decrease wild-type IDH activity in cells of the cancer, and wherein the cancer carries a histone H3.3 K27M mutation, and/or the cancer has low DNA methylation and/or low histone methylation; or (c) histone acetyltransferase, wherein the cancer carries a histone H3.3 K27M mutation, and/or high histone acetylation; or (d) histone deacetylase, wherein the cancer carries a histone H3.3 K27M mutation, and/or has high histone acetylation; or (e) any combination thereof.

### CROSS-REFERENCE TO RELATED APPLICATION

The present application claims the benefit of U.S. Provisional Application Ser. No. 63/032,653, filed May 31, 2020, which is hereby incorporated by reference herein in its entirety, including any figures, tables, nucleic acid sequences, amino acid sequences, or drawings.

### BACKGROUND OF THE INVENTION

Mutations in the DNA packaging and regulatory protein histone H3 and its primary sequence variants drive specific types of predominantly pediatric cancers, including the incurable high-grade brain stem gliomas known as Diffuse Intrinsic Pontine Gliomas (DIPG). Up to 90% of DIPG tumors carry the lysine 27 to methionine (K27M) mutation in histone H3 variants, usually the histone H3.3 variant. H3 K27M mutant high-grade pediatric gliomas such as DIPG do not currently have any approved therapies and are 100% fatal. Patients mostly receive experimental and/or palliative therapy until they pass away. It would be advantageous to have available a therapeutic approach that targets specific molecular pathways that are aberrant only in the mutant tumor cells but not the wild type cells, i.e., a therapy in which only the H3 mutant tumor cells would be eliminated specifically, while the normal cells carrying wild type H3 would be largely spared.

### BRIEF SUMMARY OF THE INVENTION

The invention concerns a method for treating or delaying the onset, progression, or relapse of a cancer in a human or non-human animal subject, comprising:

(a) administering an inhibitor of DNA-Dependent Protein Kinase catalytic subunit (DNA-PKcs inhibitor), and an inhibitor of Poly-ADP Ribose Polymerase (PARP inhibitor) to the subject, wherein the cancer has a histone H3 mutation (such as K27M, G34R/V/W/L, or K36M mutation) or is a homologous recombination-defective (HR-defective) cancer; or

(b) administering an inhibitor of wild-type isocitrate dehydrogenase (IDH inhibitor) to the subject, wherein the IDH inhibitor is administered in an effective amount to decrease wild-type IDH activity in cells of the cancer, and wherein the cancer carries a histone H3 K27M mutation, and/or the cancer has low DNA methylation and/or low histone methylation; or

(c) administering an inhibitor of histone acetyltransferase (HAT inhibitor) to the subject, wherein the cancer carries a histone H3.3 K27M mutation, and/or high histone acetylation; or

(d) administering an inhibitor of histone deacetylase (HDAC inhibitor) to the subject, wherein the cancer carries a histone H3.3 K27M mutation, and/or has high histone acetylation; or

(e) a combination of two or more of (a), (b), (c), or (d), wherein the inhibitors are administered to the subject simultaneously, or sequentially in any order.

Optionally, the method further includes administering ionizing radiation (IR) therapy to the subject to cause DNA strand breaks before, during, or after (a), (b), (c), (d), or (e).

Another aspect of the invention concerns compositions comprising combinations of inhibitors useful in the method of the invention, i.e., the method for treating or delaying the onset, progression, or relapse of a cancer. In some embodiments, the composition comprises an inhibitor of DNA-Dependent Protein Kinase catalytic subunit (DNA-PKcs inhibitor), and an inhibitor of Poly-ADP Ribose Polymerase (PARP inhibitor). In some embodiments, the composition comprises two or more of the following: (a) DNA-PKcs inhibitor, and/or PARP inhibitor; (b) a wild-type isocitrate dehydrogenase (IDH inhibitor); (c) a histone acetyltransferase (HAT inhibitor); and (d) histone deacetylase (HDAC inhibitor).

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIGS. 1A and 1B. Reactions catalyzed by different IDH enzymes and their normal product alpha-ketoglutarate (α-KG). (FIG. 1A) Reactions catalyzed by the wild type and mutant human IDH enzymes. Wildtype IDH enzymes convert isocitrate (ICT) to α-KG, while mutant IDH enzymes produce the oncometabolite D-2-hydroxyglutarate (D-2-HG). From Yang et al., 2012. (FIG. 1B) α-KG dependent DNA and histone demethylation reactions catalyzed by the dioxygenase family of demethylase enzymes. Adapted from Cairns & Mak, 2013.

FIG. 2. Model illustrating the increase in IDH activity and α-KG levels upon binding H3 K27M mutant histones that results in histone and DNA hypomethylation.

FIGS. 3A-3C. Histone H3 binds to and increases IDH1 activity in vitro. (FIG. 3A) Histone H3.3 associates with IDH1 in human cells. Whole Cell Extracts (WCEs) prepared from HCT116 cells transiently transfected with or without FLAG-H3.3 were IPed with H3 antibodies and processed for Western Blotting. Input IDH1 levels in WCEs confirm equal loading of samples in all the lanes. (FIG. 3B) Mutant histone H3.3 K27M binds IDH1 with a greater affinity than wild type H3.3 or H3.1. Recombinant HIS6 epitope-tagged human IDH1 and histones were used to assay their binding in vitro by first immobilizing HIS6-tagged IDH1 onto Talon cobalt metal affinity beads (GE Healthsciences) and incubating them with equal amounts of the indicated histones. Proteins bound to the beads were detected by Western blotting using antibodies specific for IDH1 and H3. (FIG. 3C) H3.3 binding to IDH1 enhances its activity. Generation of NADPH from NADP+ during the IDH1 mediated conversion of isocitrate to α-KG in the absence (IDH1) or presence of histone H3.3 was monitored spectrophotometrically at 340 nm.

FIGS. 4A-4D. Inhibition of IDH1 enhances both histone and DNA methylation levels in H3.3 K27M mutant DIPG cells. (FIG. 4A) H3.3 K27M mutant cells exhibit low levels of histone H3 K27me3 methylation. H3 K27 trimethylation levels measured from multiple Western blotting experiments performed on acid extracted histones prepared from SF188 pediatric glioblastoma carrying WT H3.3 and SF7761 pediatric DIPG tumor cells carrying the H3.3 K27M mutant is shown. The H3 K27 trimethylation levels were normalized to total histone H4 levels. (FIG. 4B) H3.3 K27M mutant cells exhibit very low levels of DNA methylation. Total DNA isolated from budding yeast Saccharomyces cerevisiae, SF188 cells with WT H3.3 and SF7761 DIPG cells with H3.3 K27M mutant was slot blotted onto positively charged nylon membrane. The level of methylated cytosines in the DNA was measured by immunoblotting using a highly specific rabbit monoclonal antibody that recognizes 5-Methyl Cytosine (RevMAb Biosciences) and was normalized to the total DNA measured by Ethidium bromide staining. Budding yeast exhibits negligible DNA methylation and serves as a negative control for antibody specificity here. Data from multiple experiments is plotted here and significant differences are indicated by p values. (FIG. 4C) Treatment with IDH1 inhibitor increases H3 K27me3 levels in H3.3 K27M mutant DIPG cells. Western blotting of acid extracted histones following a 5-day treatment with or without 504 IDH1 inhibitor (IDH1i) using the indicated antibodies was performed using standard procedures. Two biological replicates are shown and the average increase in H3 K27me3 levels in these cells following IDH1 inhibition after normalization to histone H4 levels is ˜470%. (FIG. 4D) IDH1 inhibition increases DNA methylation in H3.3 K27M mutant DIPG cells. DNA methylation in the indicated cells was measured following treatment with 504 IDH1 inhibitor for 5 days by South-Western blotting using a 5-meC antibody. Where applicable, p values are shown for statistically significant differences.

FIGS. 5A-5N. Inhibitors of mutant IDH1. AG-120 (Ivosidenib, highlighted in pink) (FIG. 5A) is the only FDA approved drug in this class so far. Many of the compounds listed here also inhibit wild type IDH1 at higher concentrations, several of which have been tested in our laboratory (highlighted in green (FIGS. 5C, 5H, 5M, and 5N)). Adapted from Urban et al. (2017).

FIGS. 6A and 6B. Patient-derived H3.3 K27M mutant DIPG cells are specifically sensitive to IDH1 inhibitors. (FIG. 6A) H3.3K27M mutant DIPG cells are sensitive to the IDH1 inhibitors, which may act in a synergistic manner with radiation treatment. Patient derived childhood glioblastoma cells carrying wild type (WT) H3.3 or the H3.3 K27M mutant were treated with or without the indicated brain penetrant IDH1 inhibitors (Cho et al., 2017; Zhao et al., 2018) and/or 3Gy IR. Cell viability was measured after 7 days (where applicable, the IDH1 inhibitor was added one day prior to IR exposure and maintained throughout the experiment). (FIG. 6B) Patient derived pediatric glioblastoma cells carrying wild type H3.3 or H3.3 G34V mutation are not sensitive to IDH1 inhibition. Viability of SF188 carrying wild type H3.3 and KNS42 cells carrying the H3.3 G34R mutant following IDH1 inhibition was measured. None of the treatments resulted in any significant differences in the survival of the wild type and H3.3 G34V mutant carrying cells, suggesting that only the H3 K27M mutant cells are specifically sensitive to IDH1 inhibitors as shown in FIG. 6A. CTL=controls treated with the dummy compound.

FIG. 7. DNA Double Strand Break (DSB) repair by Homologous Recombination (HR), classic Non-Homologous End Joining (NHEJ) and the Alternative-End Joining (A-EJ) pathways. Mutations in H3.3 block DSB repair by HR. Inhibition of DNA-PKcs by NU7441 blocks DSB repair via the classic NHEJ pathway, while inhibition of PARP using Olaparib blocks repair by the A-EJ pathway. Simultaneous use of NU7441 and Olaparib will block nearly all end-joining activity in cells, killing H3.3 mutant cells following radiation induced DNA breaks. (Adapted from Gong et al., 2013).

FIG. 8. Pre-clinical DNA-PK_{cs} inhibitors tested in our laboratory.

FIG. 9. FDA approved PARP inhibitors (PARPi) used in our laboratory.

FIGS. 10A-10H. Cells deficient in H3.3 show poor recruitment of Homologous Recombination (HR) factors to DNA damage sites and are defective in HR, exhibit elevated rates of Non-Homologous End Joining (NHEJ) and are sensitive to NHEJ inhibitors. (FIG. 10A) Poor recruitment of HR factor PALB2 (Partner and Localizer of BRCA2) tagged with the Yellow Fluorescent Protein (YFP) to laser induced DNA damage sites relative to the recruitment of a positive control, the DNA replication and repair protein Proliferating Cell Nuclear Antigen tagged with the Red Fluorescent Protein (RFP-PCNA) was observed in control Luc shRNA and H3.3 shRNA mediated H3.3 knockdown (H3.3 KD) cells by live cell microscopy. (FIG. 10B) Quantitation of data from multiple cells similar to the ones shown in FIG. 10A. n=number of cells analyzed. (FIG. 10C) The HR factor and single strand DNA binding protein RPA70 tagged with the Green Fluorescent Protein (GFP) is recruited slowly to laser induced DNA damage sites in H3.3 KD cells. (FIG. 10D) H3.3 knockdown cells are sensitive to Ionizing Radiation (IR) mediated DNA Double Strand Breaks (DSBs) only after release (Rel) into S-phase following serum starvation mediated G1 arrest prior to IR treatment. Flow cytometry was used to determine G1 arrest and release. UNT=untreated controls; n.s.=not significant. (FIG. 10E) HR mediated repair efficiency of an I-SceI endonuclease cleavage site within a GFP gene integrated in HCT116 cells (Fung and Weinstock, 2011) is severely inhibited in H3.3 KD cells. (FIG. 10F) H3.3 KD cells exhibit higher NHEJ efficiency as measured by a plasmid re-ligation assay. Briefly, a GFP-gene bearing plasmid was either left uncut or was linearized by cleavage within the GFP gene and then co-transfected along with a mCherry expressing plasmid. The ratio of cells showing GFP fluorescence following transfection with cut and uncut GFP plasmids is shown after normalizing for transfection efficiency using mCherry fluorescence. (FIG. 10G) H3.3 KD cells exhibit higher rates of mutagenic NHEJ mediated DSB repair in U2OS cells using a RFP based reporter as described previously (Bindra et al., 2013). (FIG. 10H) H3.3 knockdown cells are sensitive to NHEJ sub-pathway inhibitors such as 1 μM Nu7441 (a DNA-PKcs inhibitor) or 1 μM Olaparib (a PARP1 inhibitor).

FIGS. 11A-11C. Reconstituted and patient derived pediatric glioblastoma cells with the H3.3 G34 mutation are sensitive to NHEJ inhibition. Data from survival assays following treatment with or without 104 NU7441 and/or Olaparib and 2Gy IR as indicated. CTL=untreated control; Olap=Olaparib.

FIG. 11D. Survival of patient derived glioblastoma cells carrying wild type or mutant H3.3 following treatment with NHEJ inhibitors and radiation. Patient derived PCGBM2 cells carrying wild type H3.3 (control) or SF7761 and SUDIPG XIII cells with the H3.3 K27M mutant were pre-treated with 1 μM of NU and Olaparib (OLA) for 1 hour prior to treatment with or without 3Gy IR as indicated. Cell viability measured from 3 independent experiments are plotted here and statistically significant differences are shown.

FIGS. 12A-12B. Cells carrying the H3.3 K27M mutation exhibit higher levels of histone acetylation, and survival of childhood glioblastoma cells carrying wild type (WT) or mutant histone H3.3 following treatment with histone acetylase inhibitors (HATi). As shown in FIG. 12A, H3 K27M mutant cells have high levels of acetylation compared to normal cells carrying wild type H3. FIG. 12B shows that natural HATi (Curcumin, Garcinol and Anacardic acid) as well as synthetic HATi (C646, CPTH2) can specifically eliminate patient derived cancer cells carrying the H3 K27M mutation in a manner that was also synergistic with ionizing radiation, while sparing the majority of cells carrying either the wild type H3 or a different H3.3 G34V mutation.

FIG. 13A-13C. Patient derived H3.3 K27M mutant Diffuse Intrinsic Pontine Glioma (DIPG) cells exhibit high levels of histone acetylation and can be specifically eliminated following treatment with histone deacetylase inhibitors (HADCi). (FIG. 13A) H3.3 K27M mutant DIPG cells exhibit high levels of acetylation that can be enhanced even further to cytotoxic levels upon treatment with HDACi. Patient derived glioblastoma cells carrying either wild type (WT) H3.3 or the H3.3 K27M mutant were either left untreated (UT) or treated with 75 nM Vorinostat (Vor) for 16 hours before harvesting them, acid extracting total histones and processing them for Western blotting using antibodies specific to the indicated acetylated (Ac) histones. Ponceau staining of the total histones is shown as a loading control. (FIG. 13B) Treatment of cells with HDACi specifically kills the H3.3 K27M mutant DIPG cells, while largely sparing the cells carrying WT H3.3. The indicated cells were either left untreated or treated with 75 nM of the FDA approved HDACi Vorinostat (Vor; trade name Zolinza) for 7 days before counting the surviving cells. The structure of Vor is also shown. (FIG. 13C) The cytotoxic effects of HDACi synergizes with radiation in specifically eliminating H3.3K27M mutant DIPG cells. The indicated cells were either left untreated or treated with a very low dose of Vor (10 nM) either with or without a low 1Gy dose of radiation and surviving cells were counted one week later. Error bars represent standard deviation and significant differences between treatments are indicated by the p values.

FIG. 14. Breast cancer cells deficient in homologous recombination (HR) due to mutations in the BRCA1 gene are very sensitive to inhibition of Non-Homologous End Joining (NHEJ) pathways by simultaneous treatment with both DNA-PKcs and PARP inhibitors. The indicated wild type (WT) or BRCA1 mutant breast cancer cells were either left untreated or treated with the DNA-PKcs inhibitor NU7441 (NU, 1 μM) or the FDA approved PARP inhibitor Talazoparib (Talazo, 50 nM) and surviving cells were counted one week later. Error bars represent standard deviation. This data suggests that the use of lower doses of Talazoparib will allow for better visualization of the synergistic effect on cell killing when combined with NU7441, which is predicted to increase even further when combined with radiation to cause DNA strand breaks.

FIG. 15. Pancreatic cancer cells deficient in homologous recombination (HR) due to mutations in the BRCA2 gene are very sensitive to inhibition of Non-Homologous End Joining (NHEJ) pathways by simultaneous treatment with both DNA-PKcs and PARP inhibitors in the presence of radiation. The indicated wild type or BRCA2 mutant pancreatic cancer cells were either left untreated or treated with the DNA-PKcs inhibitor NU7441 (Nu; 1 μM), a very low dose of the FDA approved PARP inhibitor Talazoparib (10 nM), either with or without a low 1.5GY dose of radiation to cause DNA stand breaks and surviving cells were counted one week later. Error bars represent standard deviation.

### DETAILED DESCRIPTION OF THE INVENTION

The present invention concerns a method for treating or delaying the onset, progression, or relapse of a cancer in a human or non-human animal subject, the method selected from among:

(a) administering an inhibitor of DNA-Dependent Protein Kinase catalytic subunit (DNA-PKcs inhibitor), and an inhibitor of Poly-ADP Ribose Polymerase (PARP inhibitor) to the subject, wherein the cancer has a histone H3.3 mutation (such as K27M, G34R/V/W/L, or K36M mutation) or is a homologous recombination-defective (HR-defective) cancer; or

(b) administering an inhibitor of wild-type isocitrate dehydrogenase (IDH inhibitor) to the subject, wherein the IDH inhibitor is administered in an effective amount to decrease wild-type IDH activity in cells of the cancer, and wherein the cancer carries a histone H3.3 K27M mutation, and/or the cancer has low DNA methylation and/or low histone methylation; or

(c) administering an inhibitor of histone acetyltransferase (HAT inhibitor) to the subject, wherein the cancer carries a histone H3.3 K27M mutation, and/or high histone acetylation; or

(d) administering and inhibitor of histone deacetylase (HDAC inhibitor) to the subject, wherein the cancer carries a histone H3.3 K27M mutation, and/or has high histone acetylation; or

(e) a combination of two or more of (a), (b), (c), or (d), wherein the inhibitors are administered to the subject simultaneously, or sequentially in any order.

Thus, the method of the invention may include any one of (a), (b), (c), or (d), or, as indicated in (e), any combination of two, three, or all four of (a), (b), (c), or (d), wherein the inhibitors are administered to the subject simultaneously, or sequentially in any order. Optionally, before, during, or after (a), (b), (c), (d), or (e), the method further includes administering ionizing radiation (IR) therapy to the subject to cause DNA strand breaks

### (a) Selective Elimination of Cancer Cells Via Simultaneous Inhibition of Both Classic and Alternative Non-Homologous End Joining (NHEJ) Pathways and Induction of DNA Double Strand Breaks

In some embodiments, the method of the invention comprises (a): administering an inhibitor of DNA-Dependent Protein Kinase catalytic subunit (DNA-PKcs inhibitor), and an inhibitor of Poly-ADP Ribose Polymerase (PARP inhibitor) to the subject, wherein the cancer has a histone H3.3 mutation or is a homologous recombination-defective (FIR-defective) cancer. The histone H3.3 mutation may be, for example, a K27M, G34R/V/W/L, or K36M mutation.

The DNA-PKcs inhibitor and PARP inhibitor may be administered to the subject simultaneously, or sequentially in any order. The DNA-PKcs inhibitor and PARP inhibitor may be administered to the subject within the same composition or in separate compositions. In some embodiments, the method further comprises administering radiation therapy to the subject before, during, and/or after administering the DNA-PKcs inhibitor and PARP inhibitor. In some embodiments, the cancer comprises cancer cells bearing an H3.3 mutation, resulting in defective double strand break (DSB) repair by the homologous recombination pathway. In some embodiments, the cancer is glioblastoma comprising cancer cells bearing an H3.3 mutation. In some embodiments, the cancer is breast cancer, ovarian cancer, pancreatic cancer, prostate cancer, or melanoma bearing one or more BRCA mutations (e.g., bearing BRCA1 and/or BRCA2 mutations). Examples of DNA-PKcs inhibitors that may be used include, but are not limited to, one or a combination of compounds selected from among NU7441 (2-N-morpholino-8-dibenzothiophenyl-chromen-4-one, a.k.a. KU-57788), NU7026 (2-(morpholin-4-yl)-benzo[h]chromen-4-one), SU11752, and, AZD7648, M3814, VX-984, and CC-115 (a dual inhibitor of DNA-PK and mammalian target of rapamycin (mTOR) kinase). Examples of PARP inhibitors that may be used include, but are not limited to, one or a combination of compounds selected from among olaparib, rucaparib, niraparib, talzoparib, veliparib, BGB-290 (pamiparib), CEP 9722, E7016, and 3-aminobenzamide.

In some embodiments, the DNA-PKcs inhibitor and the PARP inhibitor are a single agent. In other embodiments, the DNA-PKcs inhibitor and the PARP inhibitor are separate agents, administered simultaneously or sequentially in any order. Additionally, ionizing radiation (IR) therapy that causes DNA double strand breaks (DSBs) can be combined with either DNA-PKcs or PARP inhibitors or both to further amplify their effects.

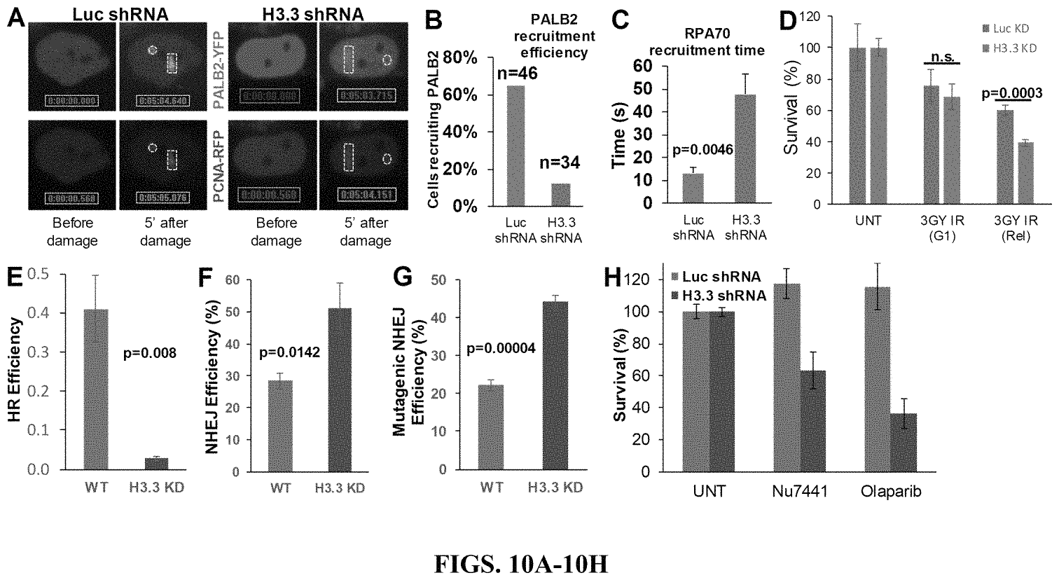

Subjects having a cancer with a histone H3.3 mutation and/or that is HR-defective can be identified by assaying a sample of cancer cells obtained from the subjects. For example, the presence of a histone H3.3 mutation (K27M, G34R/V/W/L, or K36M mutation) can be assayed using polymerase chain reaction (PCR) to amplify the H3.3 gene from the cells and performing Sanger sequencing to confirm the mutations (Grasso et al. 2015), and the presence of an HR-defect can be assessed using the assay utilized for FIG. 10E (Fung H and Weistock DM, 2011).

The inventor has determined that cancer-associated H3.3 mutations result in defective DNA double strand break (DSB) repair via the homologous recombination (HR) pathway. Based on this finding and the principle of synthetic lethality, provided is a method for selective elimination of H3.3 mutant cells, such as H3.3 mutant pediatric glioblastoma cells, using inhibitors to simultaneously inhibit or block both the classic as well as the alternative NHEJ pathways that are the only other means available to cells with defective HR to perform DSB repair. This strategy spares normal cells as they have an intact HR pathway and can use it to survive despite the inhibition of both the NHEJ pathways. This strategy for eliminating the HR-defective H3.3 mutant cancer cells by inhibiting both NHEJ pathways can also be used for the treatment of other HR defective cancers, such as breast cancer, ovarian cancer, pancreatic cancer, prostate cancer, melanoma, or other cancer carrying BRCA mutations.

In some embodiments, the method for eliminating HR defective cancer cells involving simultaneous inhibition of both the classic NHEJ pathway, using a DNA-PKcs inhibitor, and the alt-NHEJ pathway, using a PARP inhibitor, includes the administration of radiation therapy before, during, and/or after administering the DNA-PKcs inhibitor and PARP inhibitor to induce DNA double strand breaks (DSBs). This provides a highly selective tool for treating H3.3 mutant cancers such as glioblastomas, including pediatric glioblastomas.

In addition to H3.3 mutant cancers, this therapy may be used to treat a variety of cancer in which the cancer cells are defective in HR. Examples of such cancers include breast cancer carrying mutated BRCA1 and BRCA2, and other cancers carrying mutations in genes crucial for HR.

This therapy specifically eliminates HR deficient cancer cells, while sparing normal cells that have a functional HR pathway and can, therefore, use that pathway to repair DSBs and survive. Because the therapy employs inhibitors for simultaneous inhibition of two difference enzymes (DNA-PKcs and PARP1) that are crucial for the two different NHEJ pathways, it greatly reduces the risk of cancer cells developing resistance to them.

Specific mutations in the replacement histone variant H3.3 (and less commonly in H3.1) drive cancers such as disfiguring chondroblastomas and large cell tumor of the bone and lethal high-grade pediatric glioblastomas. Recent studies have implicated transcriptional defects associated with H3.3 mutations in carcinogenesis. However, transcription independent roles of H3.3 and their potential contribution to cancer have not been investigated in detail. Here we report that histone H3.3 is recruited within seconds to sites of laser induced DNA damage. Interestingly, cancer associated H3.3 mutants are defective in recruitment to DNA damage sites. Patient derived mutant H3.3 tumor cell lines as well as heterologous cells reconstituted with cancer associated H3.3 mutations or reduced levels of H3.3 accumulate high levels of endogenous DNA damage. Further, deficiency or introduction of cancer associated H3.3 mutations in fruit flies results in hypersensitivity to DNA damaging agents, suggesting that H3.3 plays an evolutionarily conserved role in DNA repair. Our data reveal a requirement for H3.3 for DNA repair by the homologous recombination (HR) pathway and suggest that DNA repair defects associated with H3.3 mutations are likely to contribute to carcinogenesis. Consistent with this idea, we successfully used a synthetic lethality (Nijman, 2011) approach involving simultaneous inhibition of both classic and alternative non-homologous end joining (NHEJ) combined with DNA double strand break (DSB) induction to selectively kill patient derived H3.3 mutant pediatric glioblastoma cells, while wild type cells with functional HR were largely spared. This therapeutic strategy could be applied broadly for the elimination of a variety of cancer cells defective in HR.

Two molecules each of canonical core histones H4, H3, H2A and H2B form an octameric protein core around which 147 base pairs of DNA is wrapped to form a nucleosome (Richmond and Davey, 2003). In most eukaryotes, one molecule of linker histone H1 is believed to bind close to the entry and exit sites of the DNA around the nucleosome core particle to form the nucleosome (Brown, 2003). This structure is repeated to package all DNA into filaments called chromatin, which then regulates access to the genetic information contained in DNA. All aspects of DNA metabolism, including DNA repair are modulated by chromatin structure. Chromatin structure in turn is regulated by posttranslational histone modifications and non-allelic primary sequence variants of the canonical histones that occur at specific loci or under certain conditions (Marzluff et al., 2002). Canonical histones H3.1 and H3.2 is deposited genome-wide during S-phase, while the replacement variant H3.3 (which differs from H3.1 and H3.2 at 5 and 4 of 135 amino acid residues, respectively) is deposited throughout the cell cycle in transcriptionally active regions, where it forms unstable nucleosomes relative to canonical H3 variants (Henikoff et al., 2009; Jin and Felsenfeld, 2007; Szenker et al., 2011; Talbert & Henikoff, 2010).

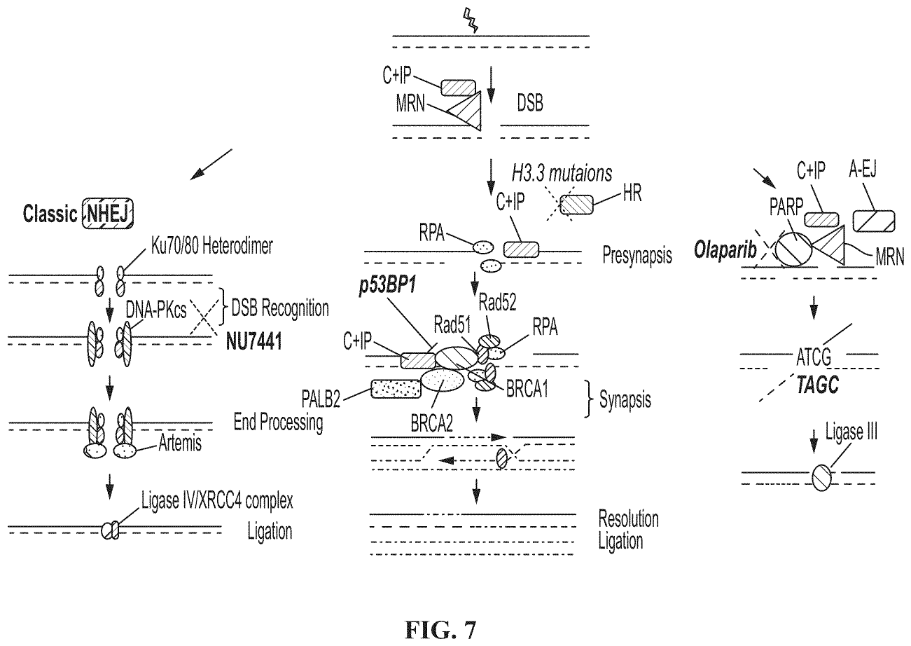

Primary brain tumors account for about 20% of childhood cancers and are a leading cause of cancer related mortality in children (Fontebasso et al., 2013). The most common type of malignant brain tumor is glioblastoma, which arises from glial cells that form the supportive tissue of the brain and is associated with very poor prognosis. Following whole genome sequencing, H3.3 K27M and G34R/V mutations were found in over a third of all the non-brain stem pediatric glioblastomas and over 80% of pediatric DIPG (Diffuse Intrinsic Pontine Glioma) tumors that are localized in the brain stem and hence cannot be surgically excised and result in the worst outcomes (Schwartzentruber et al., 2012; Wu et al., 2012; FIG. 7). Additionally, mutations in the ATRX/DAXX proteins that assemble H3.3 onto DNA were found in at least a third of the pediatric glioblastomas, including all the samples carrying the G34R/V mutations. Since then, the methylation mimetic K27M mutation has been shown to inhibit the activity of PRC2 (Polycomb Repressive Complex 2) that methylates H3K27, resulting in reduced levels of the repressive H3K27 trimethylation, which in turn leads to aberrant transcription (Chan et al., 2013; Lewis et al., 2013). On the other hand, the G34R/V mutation was shown to alter the H3K36 trimethylation pattern, thereby affecting transcription and upregulating the expression of a potent oncogene MYCN (Bjerke et al., 2013). More recently, driver K36M mutations in H3F3B and G34W/L mutations in H3F3A genes encoding H3.3 were reported in nearly all cases of chondroblastomas and large cell tumors of the bone that occur primarily in children and young adults (Behjati et al., 2013). Further, a potential tumor suppressor ZMYND11 was shown to specifically bind to H3.3 K36me3 and function as a transcriptional co-repressor (Wen et al., 2014). Hence, based on these studies, transcriptional defects caused by the H3.3 mutations have been suggested to be the major drivers of pediatric cancers. However, the studies so far do not provide any insight as to why these H3.3 mutations result in very specific types of tumors primarily in children and young adults. Given the fundamental regulation of chromatin structure by histone H3.3, it is likely that transcription independent roles of H3.3 in chromatin may also contribute to protection from cancer. Consistent with this idea, a few recent studies have suggested additional roles for H3.3 in DNA repair and genome maintenance (Frey et al., 2014; Jang et al., 2015; Yang et al., 2013; our data shown here). Together, these studies suggest that transcriptional defects may drive cancer in H3.3 mutant cells, although transcription independent roles of H3.3 and their potential contribution to tumorigenesis have not been investigated.

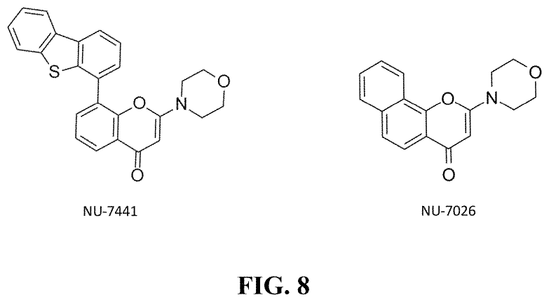

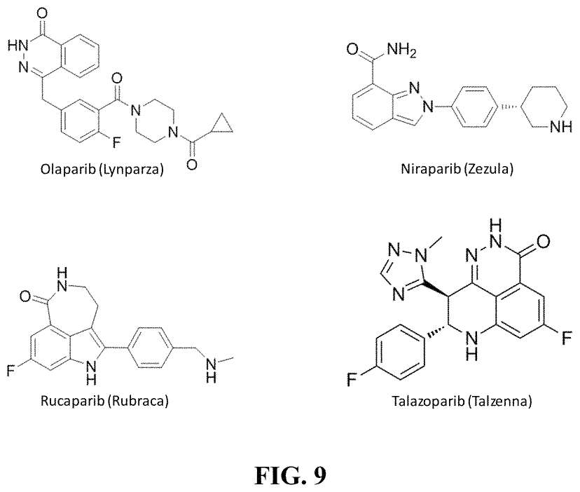

Our DNA is constantly under attack by various physical and chemical agents from both outside and within the cells (Harper and Elledge, 2007). Agents from outside the cell include ultraviolet (UV) light in the sunlight, ionizing radiation such as X-rays, toxic chemicals in the environment, etc. Agents within the cell are usually toxic byproducts that are generated in small amounts during normal cellular metabolism, such as free radicals. These agents cause a variety of damage to the DNA that the cells need to repair efficiently. A DNA double strand break is the most drastic insult that a genome can potentially suffer. A single unrepaired DSB is lethal for the cell (Bennett et al., 1996). Hence, efficient repair of a DSB is crucial for the viability of the cell. DSB repair can occur via two different pathways, NHEJ and HR (FIG. 7; Gong et al., 2013). During DSB repair via the classic or canonical NHEJ pathway, the broken ends are stabilized by the binding of the KU70/80 heterodimer which leads to the recruitment of DNA-PKcs (DNA-dependent Protein Kinase catalytic subunit) and the broken ends essentially religated by DNA Ligase IV, either with or without minimal end processing (FIG. 7; Ciccia and Elledge, 2010). If the broken ends have been processed by nucleases such as Artemis, repair by NHEJ may be inaccurate. Inhibition of DNA-PKcs with small molecule inhibitors such as NU7026 or NU7441 blocks DSB repair by the classic-NHEJ pathway (Willmore et al., 2004; Zhao et al., 2006; FIG. 8). In fact, classic NHEJ is also known as d-NHEJ for its dependence on DNA-PKcs. A parallel NHEJ-related microhomology dependent Alternative-End Joining (A-EJ or alt-NHEJ; aka b-NHEJ for backup NHEJ) pathway for DSB repair also exists in cells and requires the Poly-ADP Ribose Polymerases (PARPs) and end resection by the nuclease activities of the MRN (MRE11-RAD50-NBN) complex and CtIP (CTBP Interacting Protein) to expose short homologous sequences of 2-4 base pairs prior to ligation by Ligase III. PARP inhibition using FDA (Food and Drug Administration) approved anti-cancer drug Olaparib (FIG. 9) blocks DSB repair via the A-EJ pathway. Hence, a combination of PARP and DNA-PKcs inhibitors should block DSB repair via all the end-joining pathways (FIG. 7).

The HR pathway of DSB repair is more complex than end-joining pathways and requires extensive homologous DNA sequences which are invaded by the broken ends and copied for accurate repair. A large number of factors such as helicases are involved in HR which begins with extensive processing of the broken ends by the MRN complex and CtIP, resulting in the generation of 3′ single strand overhangs which are immediately coated by the single strand DNA binding Replication Protein A (RPA) (FIG. 7; Ciccia and Elledge, 2010). BRCA1 (Breast Cancer Susceptibility Protein 1) stimulates resection by CtIP in S and G2 phases of the cell cycle and interacts with BRCA2 (Breast Cancer 2) and its partner PALB2 (Partner And Localizer of BRCA2) that recruit RAD51 to the resected single stranded DNA ends to form nucleoprotein filaments. The Rad51 coated filament undertakes a homology search, invades the donor DNA molecule containing a suitable homologous sequence and initiates the strand exchange reaction. New DNA is synthesized to extend the single stranded regions and fill in the gap between the two broken DNA ends using the donor DNA as the template. After DNA synthesis, the crossover points (Holliday Junctions) can be resolved by a number of resolvase complexes, following which the broken ends are ligated to give rise to an accurately repaired DNA which is identical in sequence to the donor DNA in the vicinity of the initial break. The NHEJ pathways for DSB are predominant during the G1 phase of the cell cycle when extensive DNA resection is inhibited, whereas repair by HR is prominent during S and G2 phases of the cell cycle when homologous sequences are available on sister chromatids for repair by HR and extensive end resection is feasible.

Repair by both HR and NHEJ require that a number of factors have direct access to the DNA at the site of the lesion for binding and effecting repair. Since the genomic DNA of all eukaryotes is packaged into chromatin, changes in chromatin structure or composition could potentially affect DSB repair, especially for repair by HR which involves extensive stretches of chromatin. Indeed, DSB repair by HR involves extensive disruption of chromatin structure on either side of the break in yeast and chromatin remodeling machines are recruited to DSBs, presumably to open up the chromatin structure and facilitate repair (Tsukuda et al., 2005). Although chromatin dynamics at mammalian DNA breaks have not been studied in detail, it is very likely that the chromatin is significantly altered at DSBs to facilitate access to repair factors. These chromatin alterations may include disruption of pre-existing histones and recruitment of histone variants with unique properties such as the ability to form less stable nucleosomes compared to the canonical histones. Consistent with this idea, several histone variants have been shown to be recruited to DNA damage sites (Arimura et al., 2013; Sansoni et al., 2014; Xu et al., 2012), including histone H3.3 (Yang et al., 2013; our data shown here).

In the studies described herein, we show the histone H3.3 is recruited rapidly to DNA damage sites and is required for efficient HR mediated DNA repair. Deficiency of H3.3 results in a compensatory upregulation of end-joining pathways for DSB repair and accumulation of endogenous DSBs. Based on these studies, we propose that a crucial role of H3.3 in HR-mediated DNA repair contributes to genome stability and thereby in cancer prevention. We propose that DNA repair is inefficient without functional H3.3 and mutations in H3.3 lead to the accumulation of genomic alterations at an accelerated rate, eventually resulting in cancer at an early age. More importantly, we demonstrate that a synthetic lethality (Nijman, 2011) based strategy can be used successfully to specifically eliminate the HR-defective H3.3 mutant cancer cells in vitro. Future research will determine if this strategy can be employed in the clinic.

Current chemotherapeutic treatment options for H3.3 mutant childhood glioblastomas are ineffective and surgery is often precluded due to the diffuse nature of the tumor and the involvement of the brain stem in many cases, resulting in 100% fatality. On the other hand, treatment of chondroblastomas and large cell tumor of the bone often requires surgical amputation of limbs resulting in lifelong disability. The recently proposed therapeutic option that targets the epigenetic and transcriptional alterations in H3.3K27M mutant cells involves inhibiting the H3K27 histone demethylase JMJD3 (Hashizume et al., 2014). However, this strategy works only for H3.3K27M tumors and is likely to affect normal cells that require JMJD3 function as well. In contrast, the approach of the invention targets NHEJ pathways concomitant with DSB induction as a therapeutic strategy for all the three different cancer associated H3.3 mutations (K27M, G34R/V/W/L and K36M), since our data suggest that all of them are defective in HR-mediated DNA repair. Further, our strategy would spare normal cells as they would be able to use their functional HR pathway for DSB repair and survive.

Currently, the FDA approved PARP inhibitor Olaparib is being used with mixed results for the treatment of specific HR defective cancers, such as BRCA1 or BRCA2 mutated ovarian and breast cancers. However, Olaparib only inhibits the alt-NHEJ pathway (FIG. 7) and single-strand break repair via the base excision repair pathway, leaving the classic NHEJ pathway functional which can still repair DSBs in HR-defective tumor cells, thereby allowing some of these cancer cells to survive. In contrast, our strategy would involve simultaneously inhibiting both alt- and classic-NHEJ pathways for treating HR defective tumors and this should be much better at killing HR-defective cancer cells compared to PARP inhibitors alone.

Finally, Olaparib treatment alone has also been shown to result in Olaparib-resistant tumors over time (Barber et al., 2013; Jaspers et al., 2013; Xu et al., 2015). In contrast, our approach involving the use of two different inhibitors to block both classic and alt-NHEJ in HR defective cancer cells would greatly minimize the generation of resistant tumors as simultaneous acquisition of mutations that make a tumor cell resistant to both the drugs would be a much rarer event.

Overall, our data suggest that NHEJ inhibitors are effective in eliminating HR deficient H3.3 mutant tumors and can be effective in the clinic. In fact, these results also suggest that simultaneous inhibition of both classic and alternative NHEJ pathways is much more effective in eliminating HR defective cancer cells than the use of PARP inhibitors alone, which only block the alternative NHEJ pathway. Not surprisingly, monotherapy with PARP inhibitors quickly results in the development of resistant tumors and as such PARP inhibitors have not yet lived up to their promise in treating a variety of cancers. Combining PARP inhibitors with an inhibitor of classic NHEJ, such as the DNA-PKcs inhibitor NU7441 will not only kill HR defective cancer cells more effectively, but will also reduce the risk of developing resistance since it is much harder to acquire mutations that that will provide resistance to two different drugs targeting two different pathways simultaneously.

### (b) Selective Elimination of Cancer Cells Carrying H3.3 K27M Mutation Using Inhibitors of Wild-Type Isocitrate Dehydrogenase Enzyme

In some embodiments, the method of the invention comprises (b): administering an inhibitor of wild-type isocitrate dehydrogenase (IDH inhibitor) to the subject, wherein the IDH inhibitor is administered in an effective amount to decrease wild-type IDH activity in cells of the cancer, and wherein the cancer carries a histone H3.3 K27M mutation, and/or the cancer has low DNA methylation and/or low histone methylation. Optionally, the method further comprises administering radiation therapy to the subject before, during, and/or after administering the IDH inhibitor to the subject.

In some embodiments, depending on the concentration, the wild-type IDH inhibitor inhibits or blocks both wild-type IDH and mutant IDH. In other embodiments, the wild-type IDH inhibitor is specific or selective such that it does not also inhibit mutant IDH. The wild-type IDH inhibitor may inhibit, for example, wild-type IDH1, IDH2, or both IDH1 and IDH2. The wild-type IDH inhibitor may include, for example, IDH305, GSK864, or a combination thereof.

In some embodiments, the cancer to be treated is a glioblastoma or chondroblastoma.

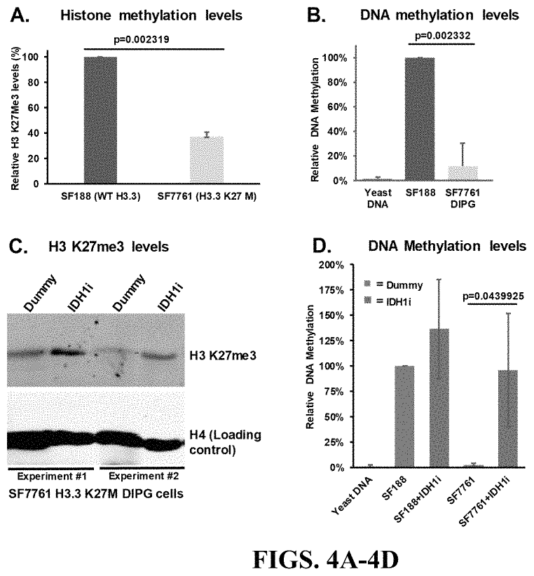

Subjects having a cancer with a histone H3.3 K27M mutation, low DNA methylation, and/or low histone methylation can be identified by assaying a sample of cancer cells obtained from the subjects. For example, the presence of a histone H3.3 mutation can be assayed using PCR to amplify the H3.3 gene from the cells and performing Sanger sequencing to confirm the mutations (Grasso et al. 2015). An assay such as a Dot Blot may be used to identify cancer cells with low DNA methylation, such as that used for FIGS. 4B and 4D. Instead of using an antibody to recognize another protein, an antibody to recognize methylated DNA antigen is used (Liu M Y et al., 2016). An assay may be used to identify cancer cells having low histone methylation using a Western blot assay, such as that used for FIGS. 4A and 4C (Bender et al., 2013; Chan et al., 2013, Lewis et al., 2013). Cancers such as pediatric glioblastomas driven by the histone H3.3 K27M mutation exhibit very low levels of DNA and histone methylation (particularly at the H3 K27 residue) and are refractory to currently available therapeutic strategies. However, it is possible that alleviation of the low DNA and histone methylation in these cancer cells, such as by pharmaceutical intervention, may sensitize them to killing by standard ionizing radiation (IR) treatment. The inventor has devised a strategy for the selective elimination of these H3.3 K27M mutant cancer cells relative to normal cells carrying wild-type H3.3. This strategy involves the use of inhibitors of wild-type IDH enzymes to block the conversion of isocitrate to alpha-ketoglutarate (α-KG), thereby inhibiting the demethylation reactions carried out by the dioxygenase family of demethylating enzymes that require α-KG for activity. This will increase the DNA and histone methylation levels in the H3.3 K27M cancer cells. Optionally, these cells can then be selectively eliminated by radiation therapy. The method of the invention is likely to be superior to therapeutic approaches that inhibit histone methylase alone and should thus result in greater tumor cell killing.

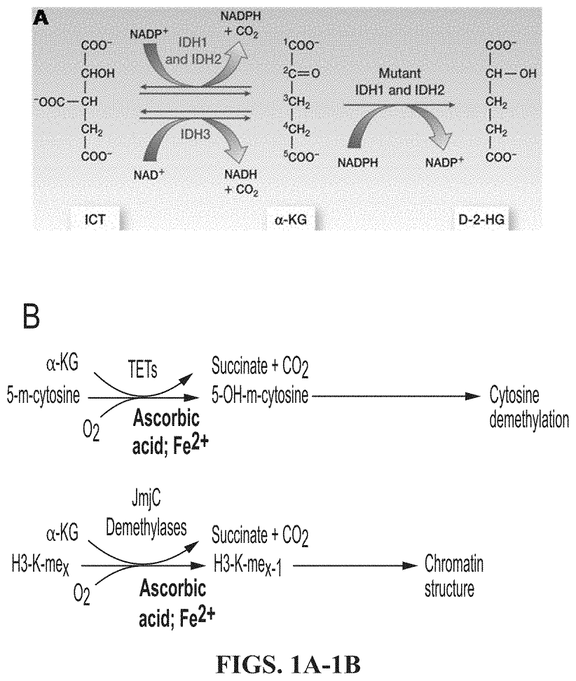

IDH are evolutionarily conserved enzymes involved in converting isocitrate to alpha-ketoglutarate (α-KG) in the Kreb's tricarboxylic acid (TCA) cycle (FIG. 1A). α-KG, in turn, is an essential co-factor for the dioxygenase family of enzymes, which includes both histone and DNA demethylases that regulate methylation marks on chromatin (Lu et al., 2012; Shih & Levine, 2012). While many cases of pediatric glioblastomas are caused by mutations in histone H3.3, a majority of adult glioblastomas are associated with mutations in IDH1 and IDH2, two of the three such enzymes found in humans (Yan et al., 2009). Specific “gain of function” point mutations in IDH1 and IDH2 alter their catalytic properties such that α-KG production is inhibited while the oncometabolite D-2-hydroxyglutarate (D-2-HG) accumulates (Dang et al., 2009; Losman et al., 2013; FIG. 1A). Although the exact process by which D-2-HG promotes carcinogenesis is unknown, it presumably competes with α-KG and inhibits the activities of α-KG dependent dioxygenases, thereby reducing demethylation reactions. Consistent with this idea, adult gliomas carrying IDH mutations are associated with a hypermethylation phenotype (Turcan et al., 2012). Tremendous efforts are currently underway to develop highly specific small molecule inhibitors to block the activity of mutant IDH1 and IDH2 for use in treatment of IDH mutant cancers and the initial results are very promising (Davis et al., 2014; Losman et al., 2013; Rohle et al., 2013; Wang et al., 2013). A byproduct of these inhibitor screens is the discovery of inhibitors of wild type IDH1 and IDH2, which are usually not studied any further as there is no need for drugs that can inhibit the wild type versions of these enzymes that are involved in the normal functioning of cells.

Primary brain tumors account for about 20% of childhood cancers and are a leading cause of cancer related mortality in children (Fontebasso et al., 2013). The most common type of malignant brain tumor is glioblastoma, which arises from glial cells that form the supportive tissue of the brain and is associated with very poor prognosis. Following whole genome sequencing, H3.3 K27M and G34R/V mutations were found in over a third of all the non-brain stem pediatric glioblastomas and nearly 80% of pediatric Diffuse Intrinsic Pontine Glioma (DIPG) tumors. DIPG tumors are localized in the brain stem and hence cannot be surgically excised, resulting in 100% fatality since there are no approved therapies available (Schwartzentruber et al., 2012; Wu et al., 2012). The methylation mimetic K27M mutation has been shown to sequester and inhibit the activity of PRC2 complex, which methylates H3 K27, resulting in reduced levels of the repressive H3 K27me3, which in turn leads to aberrant transcription and is believed to drive tumor formation (Chan et al., 2013; Lewis et al., 2013). However, a weakness in these studies is that the sequestration of PRC2 by H3.3 K27M does not explain DNA hypomethylation observed in the same cells (Bender et al., 2013; Venneti et al., 2013). Further, these studies do not provide any insight as to why these H3.3 mutations result in specific tumors primarily in children.

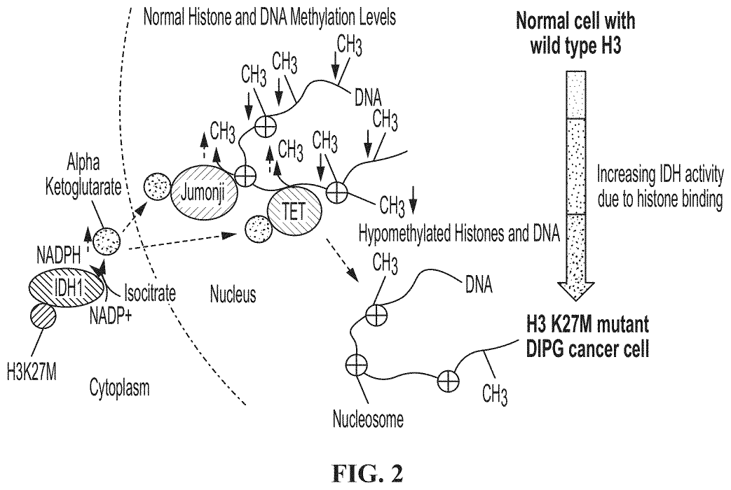

The inventor's premise is that additional non-transcriptional roles of H3.3 contribute to protection from cancer. Indeed, a few recent studies have suggested novel roles for H3.3 in genome maintenance (Frey et al., 2014; Jang et al., 2015; Yang et al., 2013; our unpublished data). Here, we show that the H3.3 K27M mutation may promote DNA and histone demethylation by binding to IDH1 and enhancing its activity leading to high α-KG levels in the cells, which in turn drives excessive dioxygenase mediated demethylation reactions, eventually resulting in the hypomethylation of both DNA and histones (FIG. 2). More importantly, we also show that IDH1 inhibition can reverse this hypomethylation, thereby potentially rendering these cells amenable to treatment using well established cancer therapies such as radiotherapy. Hence, we have now found a potential use for inhibitors of wild type IDH enzymes as they may provide us with a therapeutic strategy for childhood glioblastomas carrying the H3.3 K27M mutation that exhibit a hypomethylation phenotype.

Apart from causing pediatric glioblastomas, H3.3 mutations also drive cartilage tumors known as chondroblastomas primarily in children and young adults (Behjati et al., 2013). Interestingly, IDH1 and IDH2 mutations have been found to be associated with certain types of cartilage tumors as well (Amary et al., 2011; Suijker et al., 2015), prompting us to speculate that H3.3 and IDH mutant tumors may have more molecular features and aberrant pathways in common. Hence, the results herein are likely to provide new insights into the potential connections between the molecular pathways that give rise to both adult and pediatric glioblastomas, as well as other H3.3 and IDH mutant malignancies.

### (c) Use of Histone Acetylase Inhibitors for Treating Histone H3.3 K27M Mutant Cancers and Other Cancers Exhibiting High Levels of Histone Acetylation

In some embodiments, the method of the invention comprises (c): administering an inhibitor of histone acetyltransferase (HAT inhibitor or HATi) to the subject, wherein the cancer carries a histone H3.3 K27M mutation, and/or high histone acetylation. In some embodiments, the HAT inhibitor is a natural compound, such as curcumin, garcinol, or anacardic acid. In other embodiments, the HAT inhibitor is a synthetic compound, such as C646 or CPTH2. In some embodiments, the HAT inhibitor (natural or synthetic) is administered to the subject orally as an edible food or beverage. Optionally, the method further comprises administering radiation therapy to the subject before, during, and/or after administering the HAT inhibitor.

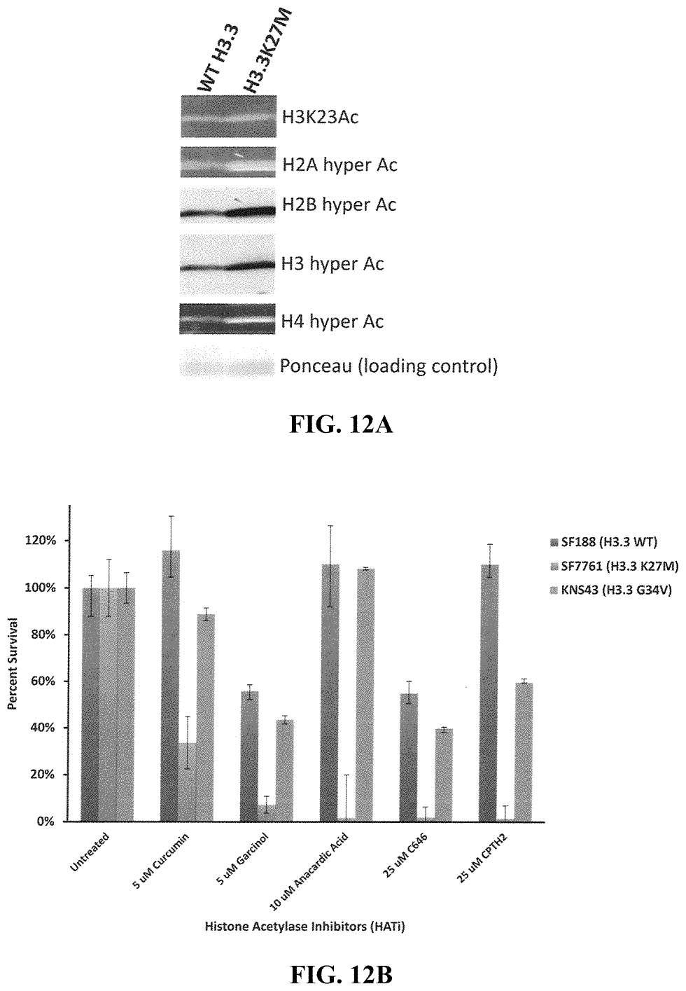

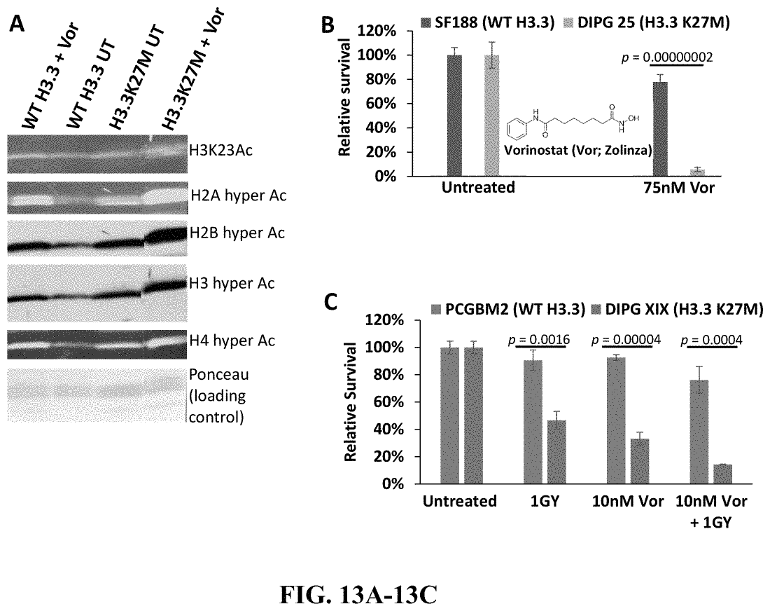

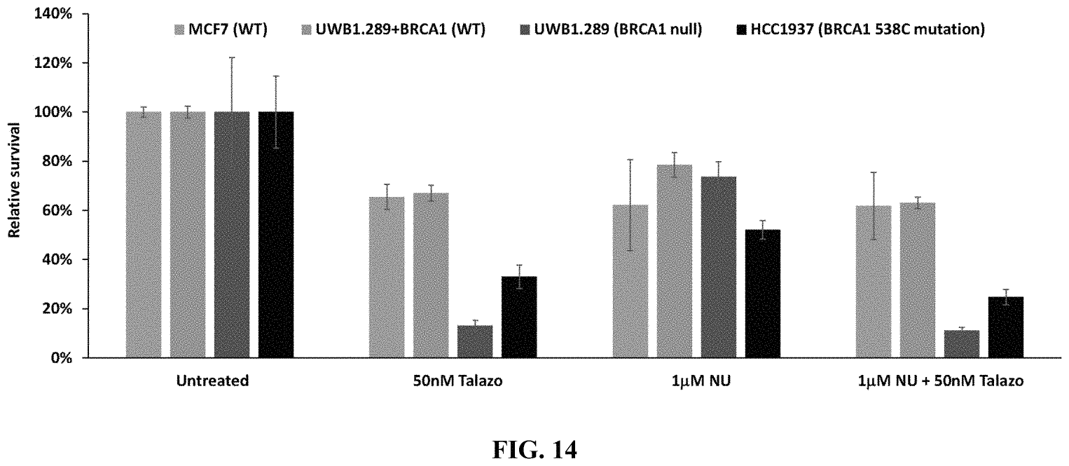

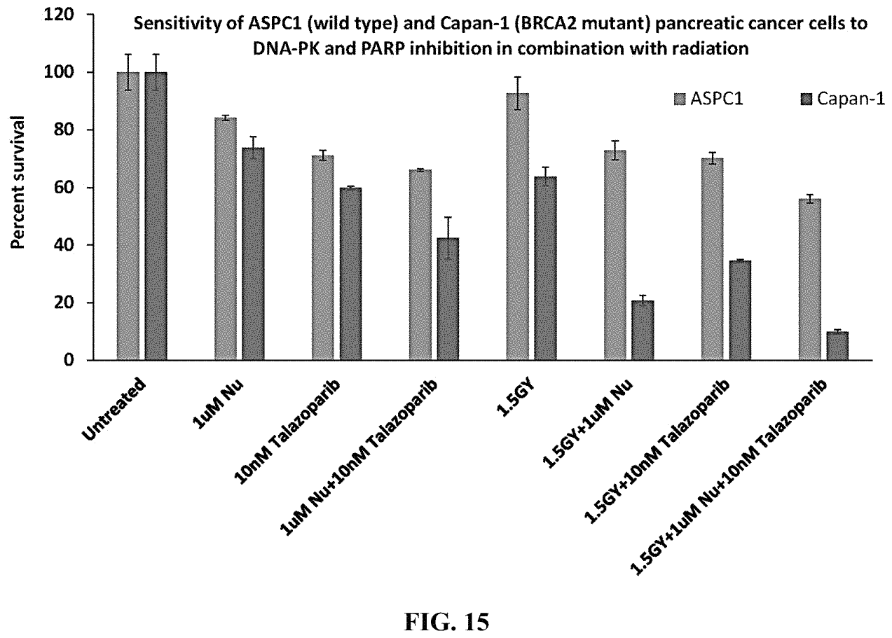

Subjects having a cancer with a histone H3.3 K27M mutation and/or high histone acetylation can be identified by assaying a sample of cancer cells obtained from the subjects. For example, the presence of a histone H3.3 mutation can be assayed using PCR to amplify the H3.3 gene from the cells and performing Sanger sequencing to confirm the mutations (Grasso et al. 2015). An assay such as a Western Blot may be used to identify cancer cells having high histone acetylation, such as that used for FIGS. 12A and 13A (Chan et al. 2013; Lewis et al. 2013).

Mutations in the DNA packaging and regulatory protein histone H3 drives specific types of predominantly pediatric cancers, including DIPG. Up to 90% of DIPG tumors carry the lysine 27 to methionine (K27M) mutation in histone H3 variants, usually the histone H3.3 variant. Cancer cells carrying the H3 K27M mutation are known to have very low levels of repressive epigenetic marks such as H3 K27 trimethylation (H3 K27me3) as well as low levels of DNA methylation. Since the levels of repressive methylation on histones and DNA are generally anti-correlated with the levels of activating epigenetic marks such as histone acetylation, the inventor hypothesized that the H3 K27M mutant cancer cells would have higher than normal levels of histone acetylation.

Cancer cells with high levels of histone acetylation are likely to be addicted to the high acetylation levels which would be necessary for driving the expression of genes required for carcinogenesis and keeping the cancer cells alive. Hence, the inventor hypothesized that cancer cells having high levels of histone acetylation can be eliminated by treating them with natural or artificial compounds that block the activity of histone acetylase (HAT) enzymes. The inventor has tested both of these hypotheses and found them to be correct. H3 K27M mutant cells indeed have high levels of acetylation compared to normal cells carrying wild type H3 (FIG. 12A). Furthermore, the inventor determined that natural (Curcumin, Garcinol and Anacardic acid) as well as synthetic (C646, CPTH2) HAT inhibitors can specifically eliminate patient derived cancer cells carrying the H3 K27M mutation in a manner that was also synergistic with ionizing radiation, while sparing the majority of cells carrying either the wild type H3 or a different H3.3 G34V mutation (FIG. 12B).

Based on these research findings, the inventor proposes HATi for treating H3 K27M mutant cancers, as well as other cancers that exhibit higher than normal levels of histone acetylation. Although potent synthetic HATi are not yet available, patients with currently incurable H3 K27M mutant DIPG and other cancers with high levels of histone acetylation can benefit from using the natural HATi compounds that are an integral part of the diet in several countries and are widely available as health supplements.

(d) Strategy for Treating Histone H3.3 K27M Mutant Cancers and Other Cancers Exhibiting High Levels of Histone Acetylation by Using Histone Deactylase Inhibitors to Further Elevate their Acetylation to Cytotoxic Levels

In some embodiments, the method of the invention comprises (d): administering and inhibitor of histone deacetylase (HDAC inhibitor) to the subject, wherein the cancer carries a histone H3.3 K27M mutation, and/or has high histone acetylation. Examples of HDAC inhibitors include vorinostat, valproic acid, or a combination thereof. Optionally, the method further comprises administering radiation therapy to the subject before, during, and/or after administering the HDAC inhibitor to the subject.

Subjects having a cancer with a histone H3.3 K27M mutation and/or high histone acetylation can be identified by assaying a sample of cancer cells obtained from the subjects. For example, the presence of a histone H3.3 mutation can be assayed using PCR to amplify the H3.3 gene from the cells and performing Sanger sequencing to confirm the mutations (Grasso et al. 2015). An assay such as a Western Blot may be used to identify cancer cells having high histone acetylation, such as that used for FIGS. 12A and 13A (Chan et al. 2013; Lewis et al. 2013).

As indicated above, the levels of repressive methylation on histones and DNA are generally anti-correlated with the levels of activating epigenetic marks such as histone acetylation; therefore, the inventor hypothesized that the H3 K27M mutant cancer cells would have higher than normal levels of histone acetylation. Cancer cells with high levels of histone acetylation are likely to be addicted to the high acetylation levels which would be necessary for driving the expression of genes required for carcinogenesis and keeping the cancer cells alive. Therefore, the inventor hypothesized that cancer cells that have high levels of histone acetylation can be eliminated by treating them with histone deacetylase inhibitors (HDACi) that inhibit or block the activity of the HDAC enzymes, thus leading to even higher levels of acetylation that are cytotoxic. The inventor has tested these hypotheses and found them to be correct. H3 K27M mutant cells indeed have high levels of acetylation compared to normal cells carrying wild type H3 (FIG. 13). Furthermore, it has been determined that potent HDACi such as Vorinostat (Zolinza) and Valproic Acid (Depakene) can specifically eliminate hyperacetylated cancer cells carrying the H3 K27M mutation in a manner that was also synergistic with ionizing radiation, while sparing the majority of cells carrying the wild type H3 with normal levels of acetylation.

Based on these research findings, the inventor proposes a method of using HDACi to treat H3 K27M mutant cancers, as well as other cancers that exhibit higher than normal levels of histone acetylation. There are several HDACi that are FDA approved (Zolinza, lstodax, Farydak, Beleodaq and Depakene) for treating a very narrow subtype of rare cancers. Patients with currently incurable H3 K27M mutant DIPG and other cancers with high levels of histone acetylation can benefit from the off-label use of these FDA approved HDACi.

This therapeutic strategy would be one of the first for H3 K27M mutant cancer cells that is rationale based, rather than based on a large drug screen where the mechanism of action is usually unknown. Furthermore, this approach specifically targets a molecular pathway that is aberrant only in the mutant tumor cells but not the wild type cells. Hence, the HDACi should specifically eliminate the H3 K27M mutant tumor cells while sparing normal cells with wild type H3, thus minimizing the potential for adverse effects.

### (e) Strategy for Histone H3 K27M Mutant Cancers, Such as DIPG, Using a Combination Approach

In some embodiments, the method of the invention comprises (e): a combination of two or more of (a), (b), (c), or (d), wherein the inhibitors are administered to the subject simultaneously, or sequentially in any order:

(a) administering an inhibitor of DNA-Dependent Protein Kinase catalytic subunit (DNA-PKcs inhibitor), and an inhibitor of Poly-ADP Ribose Polymerase (PARP inhibitor) to the subject, wherein the cancer has a histone H3.3 mutation (such as K27M, G34R/V/W/L, or K36M mutation) or is a homologous recombination-defective (HR-defective) cancer; or

(b) administering an inhibitor of wild-type isocitrate dehydrogenase (IDH inhibitor) to the subject, wherein the IDH inhibitor is administered in an effective amount to decrease wild-type IDH activity in cells of the cancer, and wherein the cancer carries a histone H3.3 K27M mutation, and/or the cancer has low DNA methylation and/or low histone methylation; or

(c) administering an inhibitor of histone acetyltransferase (HAT inhibitor) to the subject, wherein the cancer carries a histone H3.3 K27M mutation, and/or high histone acetylation; or

(d) administering and inhibitor of histone deacetylase (HDAC inhibitor) to the subject, wherein the cancer carries a histone H3.3 K27M mutation, and/or has high histone acetylation.

Optionally, the method of (e) further comprises administering radiation therapy to the subject before, during, and/or after administering the combination of inhibitors to the subject.

Subjects having a cancer susceptible to treatment with the combination of inhibitors in two or more of (a), (b), (c), and (d), such as a histone H3.3 mutation, can be identified by assaying a sample of cancer cells obtained from the subjects, as described above in connection with each of (a), (b), (c), and (d), individually.

Based on the inventor's recent findings and a mechanistic understanding of the alterations in specific molecular pathways due to histone H3 mutations, the invention includes four different therapeutic strategies by which these cancer cells can be specifically eliminated, while largely sparing the normal cells. First, cells carrying H3.3 mutations are defective in DNA repair via the HR pathway, making them heavily reliant on the NHEJ pathways for surviving DNA damage. This makes the H3.3 mutant cells sensitive to NHEJ inhibitors (NHEJi) combined with radiation therapy. Second, cells carrying the H3 K27M mutation have low levels of repressive epigenetic marks such as histone H3 K27 trimethylation (H3 K27me3) as well as low levels of DNA methylation. The inventor has shown that both the histone and DNA hypomethylation can be reversed using IDH inhibitors (IDHi) for therapeutic benefits. Third, the inventor has determined that H3 K27M mutant cancer cells also have high levels of histone acetylation which may be necessary for driving the expression of genes required for carcinogenesis and keeping the cancer cells alive. Hence, these cells can be eliminated by treatment with histone acetylase inhibitors (HATi) that reduce acetylation levels. Fourth, these cells can also be eliminated using histone deacetylase inhibitors (HDACi) that block the activity of the HDAC enzymes, thus leading to even higher levels of acetylation that are cytotoxic. Fifth, the highly proliferative tumor cells are known to be more sensitive to treatment with radiation due to the formation of DSBs, especially during DNA replication in S phase and the inventor shows that combining radiation with any of the four previous drug-based strategies results in synergistic effects leading to more efficient tumor cell killing. Finally, the inventor proposes to combine these four strategies for maximal therapeutic benefits using lower doses of the individual inhibitors to selectively kill the cancer cells while sparing normal cells. The combination therapeutic strategy would have the advantage of more extensive tumor cell elimination, minimal adverse effects, and minimal chances of development of resistance to the drugs.

### Compositions and Treatment

The inhibitors of the present invention (of (a), (b), (c), (d), and (e)) can be formulated into pharmaceutically acceptable salt forms or hydrate forms. Pharmaceutically acceptable salt forms include the acid addition salts and include hydrochloric, hydrobromic, nitric, phosphoric, carbonic, sulphuric, and organic acids like acetic, propionic, benzoic, succinic, fumaric, mandelic, oxalic, citric, tartaric, maleic, and the like. Pharmaceutically acceptable base addition salts include sodium, potassium, calcium, ammonium, and magnesium salts.

Administration of one or more inhibitors can be carried out in the form of an oral tablet, capsule, or liquid formulation containing a therapeutically effective amount of the active ingredient (inhibitor). Administration is not limited to oral delivery and includes intravascular (e.g., intravenous), intramuscular, or another means known in the pharmaceutical art for administration of active pharmaceutical ingredients.

Therapeutic or prophylactic application of the inhibitors, and compositions containing them, can be accomplished by any suitable therapeutic or prophylactic method and technique presently or prospectively known to those skilled in the art. The inhibitors can be administered by any suitable route known in the art including, for example, oral, intramuscular, intraspinal, intracranial, nasal, rectal, parenteral, subcutaneous, or intravascular (e.g., intravenous) routes of administration. Administration of the inhibitors of the invention can be continuous or at distinct intervals as can be readily determined by a person skilled in the art.

In some embodiments, an amount of inhibitors (e.g., 100 mg-1,000 mg) are to be administered 1, 2, 3, 4, or times per day, for 1, 2, 3, 4, 5, 6, 7, or more days. Treatment can continue as needed, e.g., for several weeks. Optionally, the treatment regimen can include a loading dose, with one or more daily maintenance doses. For example, in some embodiments, an initial loading dose in the range of 100 mg to 1,000 is administered, followed by a maintenance dose in the range of 100 mg to 1,000 mg every 12 hours for 1, 2, 3, 4, 5, 6, or 7, or more days. In some embodiments, an initial loading dose in the range of 200 mg to 600 mg is administered, followed by a maintenance dose in the range of 100 mg to 300 mg every 12 hours for a total of 9 doses.

Various cancers may be treated using the methods and compositions of the invention. In some embodiments, the cancer is a hematological cancer. In some embodiments, the cancer is a solid tumor. In some embodiments, the cancer is a breast cancer or gynecological cancer such as ovarian cancer (e.g., serous, epithelial, or endometrial). In some embodiments, the cancer is a brain tumor, such as glioblastoma. In some embodiments, the cancer is diffuse intrinsic pontine glioma (DIPG). In some embodiments, the cancer is pancreatic cancer, prostate cancer, or skin cancer (e.g., melanoma).

A non-exhaustive list of cancer types that may be treated using the compositions and methods of the invention is provided in Table 1.

TABLE 1
Examples of Cancer Types

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult (Primary) |
| Acute Myeloid Leukemia, Adult | Hepatocellular (Liver) Cancer, Childhood |
| Acute Myeloid Leukemia, Childhood | (Primary) |
| Adrenocortical Carcinoma | Hodgkin's Lymphoma, Adult |
| Adrenocortical Carcinoma, Childhood | Hodgkin's Lymphoma, Childhood |
| AIDS-Related Cancers | Hodgkin's Lymphoma During Pregnancy |
| AIDS-Related Lymphoma | Hypopharyngeal Cancer |
| Anal Cancer | Hypothalamic and Visual Pathway Glioma, |
| Astrocytoma, Childhood Cerebellar | Childhood |
| Astrocytoma, Childhood Cerebral | Intraocular Melanoma |
| Basal Cell Carcinoma | Islet Cell Carcinoma (Endocrine Pancreas) |
| Bile Duct Cancer, Extrahepatic | Kaposi's Sarcoma |
| Bladder Cancer | Kidney (Renal Cell) Cancer |
| Bladder Cancer, Childhood | Kidney Cancer, Childhood |
| Bone Cancer, Osteosarcoma/Malignant | |
| Fibrous Histiocytoma | Laryngeal Cancer |
| Brain Stem Glioma, Childhood | Laryngeal Cancer, Childhood |
| Brain Tumor, Adult | Leukemia, Acute Lymphoblastic, Adult |
| Brain Tumor, Brain Stem Glioma, | Leukemia, Acute Lymphoblastic, Childhood |
| Childhood | Leukemia, Acute Myeloid, Adult |
| Brain Tumor, Cerebellar Astrocytoma, | Leukemia, Acute Myeloid, Childhood |
| Childhood | Leukemia, Chronic Lymphocytic |
| Brain Tumor, Cerebral | Leukemia, Chronic Myelogenous |
| Astrocytoma/Malignant Glioma, | Leukemia, Hairy Cell |
| Childhood | Lip and Oral Cavity Cancer |
| Brain Tumor, Ependymoma, Childhood | Liver Cancer, Adult (Primary) |
| Brain Tumor, Medulloblastoma, | Liver Cancer, Childhood (Primary) |
| Childhood | Lung Cancer, Non-Small Cell |
| Brain Tumor, Supratentorial Primitive | Lung Cancer, Small Cell |
| Neuroectodermal Tumors, Childhood | Lymphoma, AIDS-Related |
| Brain Tumor, Visual Pathway and | Lymphoma, Burkitt's |
| Hypothalamic Glioma, Childhood | Lymphoma, Cutaneous T-Cell, see Mycosis |
| Brain Tumor, Childhood | Fungoides and Sézary Syndrome |
| Breast Cancer | Lymphoma, Hodgkin's, Adult |
| Breast Cancer, Childhood | Lymphoma, Hodgkin's, Childhood |
| Breast Cancer, Male | Lymphoma, Hodgkin's During Pregnancy |
| Bronchial Adenomas/Carcinoids, | Lymphoma, Non-Hodgkin's, Adult |
| Childhood | Lymphoma, Non-Hodgkin's, Childhood |
| Burkitt's Lymphoma | Lymphoma, Non-Hodgkin's During Pregnancy |
| Carcinoid Tumor, Childhood | Lymphoma, Primary Central Nervous System |
| Carcinoid Tumor, Gastrointestinal | Macroglobulinemia, Waldenström's |
| Carcinoma of Unknown Primary | Malignant Fibrous Histiocytoma of |
| Central Nervous System Lymphoma, | Bone/Osteosarcoma |
| Primary | Medulloblastoma, Childhood |
| Cerebellar Astrocytoma, Childhood | Melanoma |
| Cerebral Astrocytoma/Malignant | Glioma, Melanoma, Intraocular (Eye) |
| Childhood | Merkel Cell Carcinoma |
| Cervical Cancer | Mesothelioma, Adult Malignant |
| Childhood Cancers | Mesothelioma, Childhood |
| Chronic Lymphocytic Leukemia | Metastatic Squamous Neck Cancer with Occult |
| Chronic Myelogenous Leukemia | Primary |
| Chronic Myeloproliferative Disorders | Multiple Endocrine Neoplasia Syndrome, |
| Colon Cancer | Childhood |
| Colorectal Cancer, Childhood | Multiple Myeloma/Plasma Cell Neoplasm |
| Cutaneous T-Cell Lymphoma, see | Mycosis Fungoides |
| Mycosis Fungoides and Sezary | Myelodysplastic Syndromes |
| Syndrome | Myelodysplastic/Myeloproliferative Diseases |
| Endometrial Cancer | Myelogenous Leukemia, Chronic |
| Ependymoma, Childhood | Myeloid Leukemia, Adult Acute |
| Esophageal Cancer | Myeloid Leukemia, Childhood Acute |
| Esophageal Cancer, Childhood | Myeloma, Multiple |
| Ewing's Family of | Myeloproliferative Disorders, Chronic |
| Extracranial Germ Cell Tumor, | Nasal Cavity and Paranasal Sinus Cancer |
| Childhood | Nasopharyngeal Cancer |
| Extragonadal Germ Cell Tumor | Nasopharyngeal Cancer, Childhood |
| Extrahepatic Bile Duct Cancer | Neuroblastoma |
| Eye Cancer, Intraocular Melanoma | Non-Hodgkin's Lymphoma, Adult |
| Eye Cancer, Retinoblastoma | Non-Hodgkin's Lymphoma, Childhood |
| Gallbladder Cancer | Non-Hodgkin's Lymphoma During Pregnancy |
| Gastric (Stomach) Cancer | Non-Small Cell Lung Cancer |
| Gastric (Stomach) Cancer, Childhood | Oral Cancer, Childhood |
| Gastrointestinal Carcinoid Tumor | Oral Cavity Cancer, Lip and |
| Germ Cell Tumor, Extracranial, | Oropharyngeal Cancer |
| Childhood | Osteosarcoma/Malignant Fibrous Histiocytoma |
| Germ Cell Tumor, Extragonadal | of Bone |
| Germ Cell Tumor, Ovarian | Ovarian Cancer, Childhood |
| Gestational Trophoblastic Tumor | Ovarian Epithelial Cancer |
| Glioma, Adult | Ovarian Germ Cell Tumor |
| Glioma, Childhood Brain Stem | Ovarian Low Malignant Potential Tumor |
| Glioma, Childhood Cerebral | Pancreatic Cancer |
| Astrocytoma | Pancreatic Cancer, Childhood |
| Glioma, Childhood Visual Pathway and | Pancreatic Cancer, Islet Cell |
| Hypothalamic | Paranasal Sinus and Nasal Cavity Cancer |
| Skin Cancer (Melanoma) | Parathyroid Cancer |
| Skin Carcinoma, Merkel Cell | Penile Cancer |
| Small Cell Lung Cancer | Pheochromocytoma |
| Small Intestine Cancer | Pineoblastoma and Supratentorial Primitive |
| Soft Tissue Sarcoma, Adult | Neuroectodermal Tumors, Childhood |
| Soft Tissue Sarcoma, Childhood | Pituitary Tumor |
| Squamous Cell Carcinoma, see Skin | Plasma Cell Neoplasm/Multiple Myeloma |
| Cancer (non-Melanoma) | Pleuropulmonary Blastoma |
| Squamous Neck Cancer with Occult | Pregnancy and Breast Cancer |
| Primary, Metastatic | Pregnancy and Hodgkin's Lymphoma |
| Stomach (Gastric) Cancer | Pregnancy and Non-Hodgkin's Lymphoma |
| Stomach (Gastric) Cancer, Childhood | Primary Central Nervous System Lymphoma |
| Supratentorial Primitive | Prostate Cancer |
| Neuroectodermal Tumors, Childhood | Rectal Cancer |
| T-Cell Lymphoma, Cutaneous, see | Renal Cell (Kidney) Cancer |
| Mycosis Fungoides and Sezary | Renal Cell (Kidney) Cancer, Childhood |
| Syndrome | Renal Pelvis and Ureter, Transitional Cell |
| Testicular Cancer | Cancer |
| Thymoma, Childhood | Retinoblastoma |
| Thymoma and Thymic Carcinoma | Rhabdomyosarcoma, Childhood |
| Thyroid Cancer | Salivary Gland Cancer |
| Thyroid Cancer, Childhood | Salivary Gland Cancer, Childhood |
| Transitional Cell Cancer of the Renal | Sarcoma, Ewing's Family of Tumors |
| Pelvis and Ureter | Sarcoma, Kaposi's |
| Trophoblastic Tumor, Gestational | Sarcoma, Soft Tissue, Adult |
| Unknown Primary Site, Carcinoma of, | Sarcoma, Soft Tissue, Childhood |
| Adult | Sarcoma, Uterine |
| Unknown Primary Site, Cancer of | Sezary Syndrome |
| Childhood | Skin Cancer (non-Melanoma) |
| Unusual Cancers of Childhood | Skin Cancer, Childhood |
| Ureter and Renal Pelvis, Transitional Cell | Stomach Cancer |
| Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström's Macroglobulinemia | |
| Wilms' Tumor | |

Inhibitors and compositions comprising them can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive inhibitor is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject inhibitors include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of the inhibitors can also comprise suspension agents, protectants, lubricants, buffers, preservatives, and stabilizers. To provide for the administration of such dosages for the desired therapeutic treatment, pharmaceutical compositions of the invention will advantageously comprise between about 0.1% and 45%, and especially, 1 and 15% by weight of the total of one or more of the inhibitor-based on the weight of the total composition including carrier or diluent.

The inhibitors of the subject invention can also be administered utilizing liposome technology, slow release capsules, implantable pumps, and biodegradable containers. These delivery methods can, advantageously, provide a uniform dosage over an extended period of time.

The inhibitors can also be modified by the addition of chemical groups, such as PEG (polyethylene glycol). PEGylated polypeptides typically generate less of an immunogenic response and exhibit extended half-lives in vivo in comparison to polypeptides that are not PEGylated when administered in vivo. Methods for PEGylating proteins and polypeptides known in the art (see, for example, U.S. Pat. No. 4,179,337). Inhibitors can also be modified to improve cell membrane permeability. In one embodiment, cell membrane permeability can be improved by attaching a lipophilic moiety, such as a steroid, to the inhibitor. Other groups known in the art can be linked to the inhibitors.

The subject invention also concerns a packaged dosage formulation comprising in one or more packages, packets, or containers at least one inhibitor and/or composition of the subject invention formulated in a pharmaceutically acceptable dosage. The package can contain discrete quantities of the dosage formulation, such as tablet, capsules, lozenge, and powders. The quantity of inhibitor in a dosage formulation and that can be administered to a patient can vary from about 1 mg to about 5000 mg, or about 1 mg to about 2000 mg, or more typically about 1 mg to about 500 mg, or about 5 mg to about 250 mg, or about 10 mg to about 100 mg. In some embodiments, the amount is in the range of 100 mg to 600 mg, to be administered 1, 2, 3, or 4 times per day, for 2, 3, 4, 5, 6, 7 or more days.

The subject invention also concerns kits comprising in one or more containers an inhibitor of the present invention. A kit of the invention can also comprise one or more compounds, biological molecules, or drugs. In one embodiment, a kit of the invention comprises a combination of two or more inhibitors from among (a), (b), (c), and (d).

Optionally, the methods further comprise, prior to administering the inhibitor combination of inhibitors to the subject, identifying the subject as having the cancer. If the subject is identified as having the cancer, the inhibitor or combination of inhibitors can be administered to the subject as therapy. If the human subject is identified as not having a cancer, the inhibitor or combination of inhibitors can be withheld, or the inhibitor or combination of inhibitors can be administered as prophylaxis to prevent or delay the onset or relapse of the cancer, or an alternative agent can be administered to the subject. The identifying step may comprise carrying out one or both of the following: a cytology or cytopathology test of a cell sample from the subject, an assay based on the presence or level of a biomarker associated with the cancer in a biological sample from the subject (e.g., body fluid such as blood or urine, or a biopsy), or an imaging test of the subject (e.g., CT scan, MRI, nuclear scan, bone scan, PET scan, ultrasound, or x-ray).

The subject may be any age or gender. In some embodiments, the subject is a human pediatric patient (ages 0-18 years).

Optionally, the methods of the invention ((a), (b), (c), (d), and (e)) further comprise administering radiation therapy to the subject before, during, and/or after administering the inhibitor or combination of inhibitors. The radiation therapy preferably comprises an ionizing radiation selected from among: external radiation (e.g., external beam radiation therapy (EBRT or XRT)), internal radiation (e.g., brachytherapy), or systemic radiation (e.g., systemic radioisotope therapy), or a combination of two or more of the foregoing. The radiation therapy may include a fractionation regimen (e.g., hypofractionation, hyperfractionation, accelerated fractionation).

The administration of ionizing radiation (IR) therapy may be used to cause DNA strand breaks in combination with any of the strategies listed in (a), (b), (c), or (d) to amplify the effects of the drugs individually or in combination.

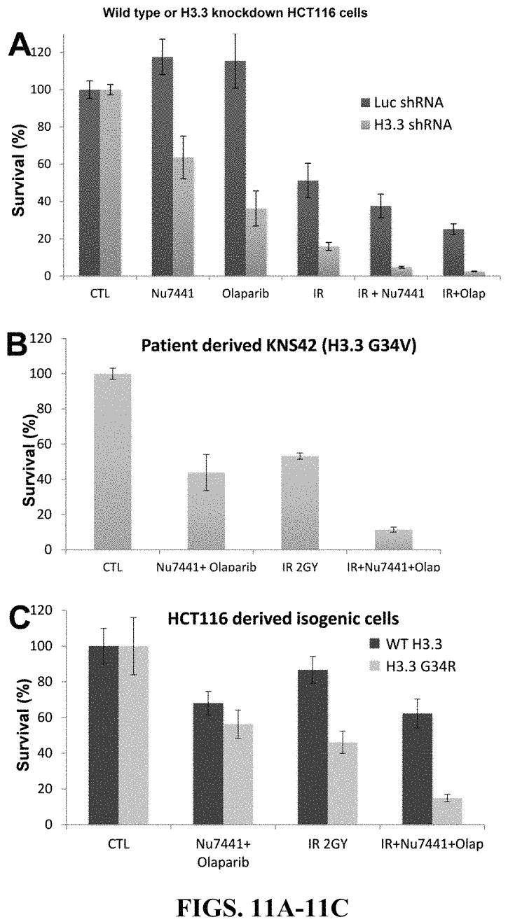

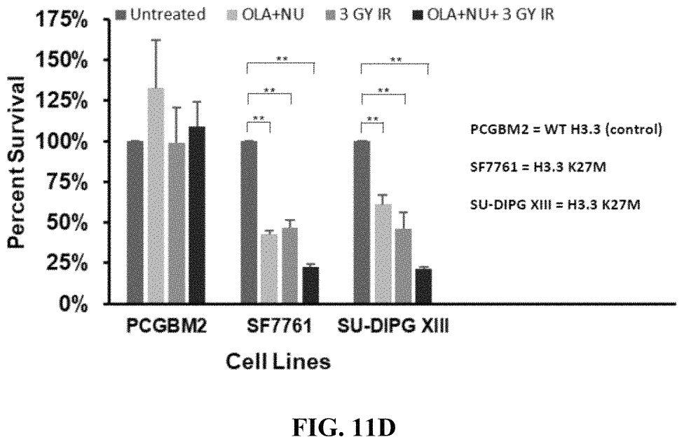

Radiation therapy by itself is an established and FDA approved treatment for a variety of cancers due to its ability to cause DSBs that need to be repaired for cell survival, failing which cells carrying unrepaired DSBs die (FIG. 10D, FIG. 11). Hence, combining radiation therapy with NHEJ inhibitors listed in (a) that block DSB repair is expected to enhance the cell killing effect of the NHEJ inhibitors, and this is exactly what we observe (FIG. 11A-C). Remarkably, IDH1 inhibition also appeared to show a synergistic effect with DNA damage caused by ionizing radiation (IR) treatment in eliminating multiple patient derived H3.3 K27M mutant DIPG cancer cells (FIG. 10A, B). This synergy between IDH1 inhibition and radiation is not surprising at all given that DNA methylation levels are known to impact the radiation sensitivity of cells and this has been getting increasing attention in recent years as a possible target for improving the efficiency of radiation therapy for cancer (Chi et al., 2018; Ou et al., 2018; Zielske, 2015; Zhu et al., 2018). Additionally, histone modifications including methylation have also implicated in modulating the efficiency of radiation therapy (Gursoy-Yuzugullu et al., 2017; Smits et al., 2014) and a histone H3 demethylase inhibitor has been reported to enhance the efficacy of radiotherapy of DIPG tumors xenografted in mice (Katagi et al., 2019). Furthermore, histones accumulate transiently upon DNA damage (Gunjan and Verreault, 2003) and the interaction between histones and IDH proteins is also enhanced upon DNA damage (data not shown). Hence, it is possible that the regulation of IDH enzymes by histones and their downstream effects on histone and DNA methylation may be physiologically important for survival following DNA damage. Finally, there are numerous reports in the literature suggesting that the effects of radiation can also be potentiated by treatment with HDACi (Karagiannis and El-Osta, 2006). Hence, we tested the combined effects of treatment with HDACi and radiation and found the H3.3 K27M mutant cells to be exquisitely sensitive to the combination, while the WT cells were largely unaffected (FIG. 13C). Remarkably, this combination treatment requires very low doses of Vor (10 nM) and radiation (1Gy) to specifically eliminate H3.3 K27M DIPG cells. Overall, combining radiation therapy with any of the therapeutic strategies listed in (a), (b), (c) or (d) can result in synergistic cytotoxic effects on H3 mutant, HR defective, hypomethylated or hyperacetylated cancer cells, leading to more efficient elimination of cancer cells, while sparing the normal cells. The radiation therapy may be single dose or fractionated, and may be targeted or non-targeted.

In some embodiments, the radiation therapy is image-guided. An image-guided device, such as the CYBERKNIFE™ device, may be used. The device combines a compact linear accelerator mounted on a robotic manipulator, and an integrated image guidance system. The image guidance system acquires stereoscopic kV images during treatment, tracks tumor motion, and guides the robotic manipulator to align the treatment beam to the moving tumor. The system is designed for stereotactic radiosurgery (SRS) and stereotactic body radiation therapy (SBRT). The system is also used for select 3D conformal radiotherapy (3D-CRT) and intensity modulated radiation therapy (IMRT).

Optionally, the methods of the invention further comprise administering one or more additional biologically active agents to the subject before, during, and/or after administration of the one or more inhibitors of the invention. In some embodiments, the additional biologically active agent includes one or more anti-cancer agents, such as a chemotherapeutic agent and/or an immunomodulatory agent such as an immune checkpoint inhibitor.

The invention includes compositions that may be used for carrying out methods of the invention. In one aspect, the composition comprises DNA-PKcs inhibitor, and a PARP inhibitor. Examples of DNA-PKcs inhibitors that may be used include NU7441 (2-N-morpholino-8-dibenzothiophenyl-chromen-4-one, a.k.a. KU-57788), NU7026 (2-(morpholin-4-yl)-benzo[h]chromen-4-one), SU11752, NK314, AZD7648, M3814, VX-984, and CC-115, or a combination of two or more of the foregoing. Examples of PARP inhibitors that may be used include olaparib, rucaparib, niraparib, talzoparib, veliparib, BGB-290 (pamiparib), CEP 9722, E7016, and 3-aminobenzamide, or a combination of two or more of the foregoing. The composition may further comprise a pharmaceutically acceptable carrier or diluent.

In another aspect, the composition comprises two or more of the following: (a) a DNA-PKcs inhibitor, and/or a PARP inhibitor; (b) a wild-type IDH inhibitor; (c) a HAT inhibitor; and (d) a HDAC inhibitor. Examples of DNA-PKcs inhibitors that may be used include NU7441 (2-N-morpholino-8-dibenzothiophenyl-chromen-4-one, a.k.a. KU-57788), NU7026 (2-(morpholin-4-yl)-benzo[h]chromen-4-one), SU11752, NK314, AZD7648, M3814, VX-984, and CC-115, or a combination of two or more of the foregoing. Examples of PARP inhibitors that may be used include olaparib, rucaparib, niraparib, talzoparib, veliparib, BGB-290 (pamiparib), CEP 9722, E7016, and 3-aminobenzamide, or a combination of two or more of the foregoing. Examples of IDH inhibitors include IDH305 and GSK864, or a combination thereof. The HAT inhibitor may be, for example, a natural compound, such as curcumin, garcinol, anacardic acid, or a combination of two or more of the foregoing, or a synthetic compound such as C646 or CPTH2, or combination thereof. Examples of HDAC inhibitors that may be used include vorinostat, valproic acid, or a combination thereof. The composition may further comprise a pharmaceutically acceptable carrier or diluent.

The invention further provides kits, including one or more inhibitors and pharmaceutical formulations, packaged into suitable packaging material, optionally in combination with instructions for using the kit components, e.g., instructions for performing a method of the invention. In one embodiment, a kit includes an amount of one or more inhibitors, and instructions for administering the inhibitor or combination of inhibitors to a subject in need of treatment on a label or packaging insert. In further embodiments, a kit includes an article of manufacture, for delivering the inhibitor or combination of inhibitors into a subject locally, regionally or systemically, for example.

As used herein, the term “packaging material” refers to a physical structure housing the components of the kit. The packaging material can maintain the components in a sterile state, and can be made of material commonly used for such purposes (e.g., paper, corrugated fiber, glass, plastic, foil, ampules, etc.). The label or packaging insert can include appropriate written instructions, for example, practicing a method of the invention, e.g., treating cancer, an assay for identifying a subject having the cancer to be treated, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, pharmaceutical compositions can be included in a container, pack, or dispenser together with instructions for administration to a subject to treat a cancer. Instructions may additionally include appropriate administration route, dosage information, indications of a satisfactory clinical endpoint or any adverse symptoms that may occur, storage information, expiration date, or any information required by regulatory agencies such as the Food and Drug Administration or European Medicines Agency for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Kits can additionally include a buffering agent, a preservative, or an agent for stabilizing the inhibitor or combination of inhibitors. The kit can also include control components for assaying for the presence of cancer, e.g., a control sample or a standard. Each component of the kit can be enclosed within an individual container or in a mixture and all of the various containers can be within single or multiple packages.

### Exemplified Embodiments

Embodiment 1. A method for treating or delaying the onset, progression, or relapse of a cancer in a human or non-human animal subject, the method selected from among:

(a) administering an inhibitor of DNA-Dependent Protein Kinase catalytic subunit (DNA-PKcs inhibitor), and an inhibitor of Poly-ADP Ribose Polymerase (PARP inhibitor) to the subject, wherein the cancer has a histone H3.3 mutation (such as K27M, G34R/V/W/L, or K36M mutation) or is a homologous recombination-defective (HR-defective) cancer; or

(b) administering an inhibitor of wild-type isocitrate dehydrogenase (IDH inhibitor) to the subject, wherein the IDH inhibitor is administered in an effective amount to decrease wild-type IDH activity in cells of the cancer, and wherein the cancer carries a histone H3.3 K27M mutation, and/or the cancer has low DNA methylation and/or low histone methylation; or

(c) administering an inhibitor of histone acetyltransferase (HAT inhibitor) to the subject, wherein the cancer carries a histone H3.3 K27M mutation, and/or has high histone acetylation; or

(d) administering an inhibitor of histone deacetylase (HDAC inhibitor) to the subject, wherein the cancer carries a histone H3.3 K27M mutation, and/or has high histone acetylation; or

(e) a combination of two or more of (a), (b), (c), or (d), wherein the inhibitors are administered to the subject simultaneously, or sequentially in any order.

Embodiment 2. The method of embodiment 1, wherein the method comprises (a).

Embodiment 3. The method of embodiment 1, wherein the method comprises (b).

Embodiment 4. The method of embodiment 1, wherein the method comprises (c).

Embodiment 5. The method of embodiment 1, wherein the method comprises (d).

Embodiment 6. The method of embodiment 1, wherein the method comprises (e).

Embodiment 7. The method of embodiment 1, wherein the method comprises (a), further comprising administering radiation therapy to the subject before, during, and/or after administering the DNA-PKcs inhibitor and PARP inhibitor.

Embodiment 8. The method of embodiment 1, wherein the method comprises (a), and wherein the cancer comprises cancer cells bearing an H3.3 mutation, resulting in defective double strand break (DSB) repair by the homologous recombination pathway.

Embodiment 9. The method of embodiment 1, wherein the method comprises (a), and wherein the cancer is glioblastoma comprising cancer cells bearing an H3.3 mutation.

Embodiment 10. The method of embodiment 1, wherein the method comprises (a), and wherein the cancer is breast cancer, ovarian cancer, pancreatic cancer, prostate cancer, or melanoma bearing one or more BRCA mutations (e.g., bearing BRCA1 and/or BRCA2 mutations).

Embodiment 11. The method of embodiment 1, wherein the method comprises (a), and wherein the cancer is any cancer exhibiting a defective homologous recombination pathway.

Embodiment 12. The method of embodiment 1 wherein the method comprises (a), and wherein the DNA-PKcs inhibitor comprises one or more compounds selected from among NU7441 (2-N-morpholino-8-dibenzothiophenyl-chromen-4-one, a.k.a. KU-57788), NU7026 (2-(morpholin-4-yl)-benzo[h]chromen-4-one), SU11752, NK314, AZD7648, M3814, VX-984, and CC-115.

Embodiment 13. The method of embodiment 1, wherein the method comprises (a), and wherein the PARP inhibitor comprises one or more compounds selected from among olaparib, rucaparib, niraparib, talzoparib, veliparib, BGB-290 (pamiparib), CEP 9722, E7016, and 3-aminobenzamide.

Embodiment 14. The method of embodiment 1, wherein the method comprises (a), and wherein the DNA-PKcs inhibitor and the PARP inhibitor are a single agent.

Embodiment 15. The method of embodiment 1, wherein the method comprises (a), and wherein the DNA-PKcs inhibitor and the PARP inhibitor are separate agents, administered simultaneously or sequentially in any order.

Embodiment 16. The method of embodiment 1, wherein the method comprises (b), further comprising administering radiation therapy to the subject before, during, and/or after administering the IDH inhibitor to the subject.

Embodiment 17. The method of embodiment 1 or 16, wherein the IDH inhibitor does not also inhibit mutant IDH.

Embodiment 18. The method of embodiment 1 or 16, wherein the method comprises (b), and wherein the IDH inhibitor inhibits wild-type IDH1, IDH2, or both IDH1 and IDH2.

Embodiment 19. The method of embodiment 1, wherein the method comprises (b), and wherein the cancer is a glioblastoma or chondroblastoma.

Embodiment 20. The method of embodiment 1, wherein the method comprises (b), and wherein the cancer is any cancer exhibiting DNA hypomethylation or histone hypomethylation, or both.

Embodiment 21. The method of embodiment 1, wherein the method comprises (b), and wherein the IDH inhibitor comprises IDH305, GSK864, or a combination thereof.

Embodiment 22. The method of embodiment 1, wherein the method comprises (c), and wherein the HAT inhibitor is a natural compound, such as curcumin, garcinol, or anacardic acid.

Embodiment 23. The method of embodiment 1, wherein the method comprises (c), and wherein the HAT inhibitor is a synthetic compound, such as C646 or CPTH2.

Embodiment 24. The method of embodiment 1, wherein the method comprises (c), further comprising administering radiation therapy to the subject before, during, and/or after administering the HAT inhibitor.

Embodiment 25. The method of embodiment 1, wherein the method comprises (c), and wherein the HAT inhibitor is administered to the subject orally as an edible food or beverage.

Embodiment 26. The method of embodiment 1, wherein the method comprises (c), and wherein the cancer is any cancer exhibiting histone hyperacetylation.

Embodiment 27. The method of embodiment 1, wherein the method comprises (d), and wherein the HDAC inhibitor comprises vorinostat, valproic acid, or a combination thereof.

Embodiment 28. The method of embodiment 1, wherein the method comprises (d), further comprising administering radiation therapy to the subject before, during, and/or after administering the HDAC inhibitor to the subject.

Embodiment 29. The method of embodiment 1, wherein the method comprises (b), and wherein the cancer is any cancer exhibiting histone hyperacetylation.

Embodiment 30. The method of embodiment 1, wherein the method comprises (a), (b), (c), (d), or (e), and wherein the cancer is diffuse intrinsic pontine glioma (DIPG).

Embodiment 31. The method of embodiment 1, wherein the method comprises (a), (b), (c), (d), or (e), and wherein the subject is a human pediatric patient (ages 0 to 18 years).

Embodiment 32. The method of embodiment 1, wherein the method comprises (a), (b), (c), (d), or (e), and wherein the subject is a human adult patient.

Embodiment 33. The method of embodiment 1, wherein the method comprises (a), (b), (c), (d), or (e), further comprising administering radiation therapy to the subject before, during, and/or after administering the inhibitor.

Embodiment 34. The method of embodiment 1, wherein the method comprises (a), (b), (c), (d), or (e), further comprising administering ionizing radiation (IR) therapy to the subject to cause DNA strand breaks.

Embodiment 35. The method of embodiment 33, wherein the radiation therapy comprises an ionizing radiation selected from among: external radiation (e.g., external beam radiation therapy (EBRT or XRT)), internal radiation (e.g., brachytherapy), or systemic radiation (e.g., systemic radioisotope therapy), or a combination of two or more of the foregoing.

Embodiment 36. The method of embodiment 33, wherein the radiation therapy comprises a fractionation regimen (e.g., hypofractionation, hyperfractionation, accelerated fractionation).

Embodiment 37. The method of embodiment 1, wherein the method comprises (a), (b), (c), (d), or (e), further comprising, prior to said administering, identifying the subject as having the cancer.

Embodiment 38. The method of embodiment 37, wherein the subject is identified by one or both of the following: a cytology or cytopathology test of a cell sample from the subject, an assay based on the presence or level of a biomarker associated with the cancer in a biological sample from the subject (e.g., body fluid such as blood or urine, or a biopsy), or an imaging test of the subject (e.g., CT scan, MM, nuclear scan, bone scan, PET scan, ultrasound, or x-ray).

Embodiment 39. A composition comprising an inhibitor of DNA-Dependent Protein Kinase catalytic subunit (DNA-PKcs inhibitor), and an inhibitor of Poly-ADP Ribose Polymerase (PARP inhibitor).

Embodiment 40. The composition of embodiment 39, wherein the DNA-PKcs inhibitor comprises one or more compounds selected from among NU7441 (2-N-morpholino-8-dibenzothiophenyl-chromen-4-one, a.k.a. KU-57788), NU7026 (2-(morpholin-4-yl)-benzo[h]chromen-4-one), SU11752, NK314, AZD7648, M3814, VX-984, and CC-115; and wherein the PARP inhibitor comprises one or more compounds selected from among olaparib, rucaparib, niraparib, talzoparib, veliparib, BGB-290 (pamiparib), CEP 9722, E7016, and 3-aminobenzamide.

Embodiment 41. The composition of embodiment 39 or 40, further comprising a pharmaceutically acceptable carrier or diluent.

Embodiment 42. A composition comprising two or more of the following:

(a) an inhibitor of DNA-Dependent Protein Kinase catalytic subunit (DNA-PKcs inhibitor), and/or an inhibitor of Poly-ADP Ribose Polymerase (PARP inhibitor);

(b) a wild-type isocitrate dehydrogenase (IDH inhibitor);

(c) a histone acetyltransferase (HAT inhibitor); and

(d) histone deacetylase (HDAC inhibitor).

Embodiment 43. The composition of embodiment 42, wherein the composition comprises (a); wherein the DNA-PKcs inhibitor comprises one or more compounds selected from among NU7441 (2-N-morpholino-8-dibenzothiophenyl-chromen-4-one, a.k.a. KU-57788), NU7026 (2-(morpholin-4-yl)-benzo[h]chromen-4-one), SU11752, NK314, AZD7648, M3814, VX-984, and CC-115; and wherein the PARP inhibitor comprises one or more compounds selected from among olaparib, rucaparib, niraparib, talzoparib, veliparib, BGB-290 (pamiparib), CEP 9722, E7016, and 3-aminobenzamide.

Embodiment 44. The composition of embodiment 42, wherein the composition comprises (b) and one or more of (a), (c), or (d), and wherein the IDH inhibitor comprises IDH305, GSK864, or a combination thereof.

Embodiment 45. The composition of embodiment 42, wherein the composition comprises (c) and one or more of (a), (b), and (d), and wherein the HAT inhibitor is a natural compound, such as curcumin, garcinol, or anacardic acid.

Embodiment 46. The composition of embodiment 42, wherein the composition comprises (c) and one or more of (a), (b), (d) and (e), and wherein the HAT inhibitor is a synthetic compound, such as C646 or CPTH2.

Embodiment 47. The composition of embodiment 42, wherein the composition comprises (d) and one or more of (a), (b), and (c), and wherein the HDAC inhibitor comprises vorinostat, valproic acid, or a combination thereof.

Embodiment 48. The composition of any one of embodiments 42-47, further comprising a pharmaceutically acceptable carrier or diluent.

### Definitions

As used herein, a subject is “in need of” a treatment if such human subject would benefit biologically, medically or in quality of life from such treatment (preferably, a human). In some embodiments, the subject has cancer and is in need of therapy. In other embodiments, the subject does not have cancer and is in need of prophylaxis to prevent or delay onset or relapse of the cancer. In some embodiments, the subject in need of prophylaxis is at risk of developing the cancer or relapse of the cancer. In some embodiments, the subject is at increased risk of developing the cancer or relapse of the cancer relative to others in the population.

As used herein, the terms “subject”, “patient”, and “individual” refer to a human or non-human animal of any age or sex. In some embodiments, the subject is a mammal (human or non-human mammal).

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment, “treat”, “treating” or “treatment” refers to alleviating or ameliorating at least one physical parameter including those which may not be discernible by the subject. In yet another embodiment, “treat”, “treating” or “treatment” refers to modulating the disease or disorder, either physically, (e.g., stabilization of a discernible symptom), physiologically, (e.g., stabilization of a physical parameter), or both. In another embodiment, “treat”, “treating” or “treatment” refers to reduction in tumor size, reduction in rate of tumor growth, delay in disease progression, and/or increase in the individual's disease-free survival (relapse-free survival). In yet another embodiment, “treat”, “treating” or “treatment” refers to prophylaxis (preventing or delaying the onset, development, progression, or relapse of the disease or disorder).

As used herein, the term “tumor” refers to all neoplastic cell growth and proliferation, whether malignant or benign, and all pre-cancerous and cancerous cells and tissues. For example, a particular cancer may be characterized by a solid mass tumor or non-solid tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography) or magnetic resonance imaging (MRI), or by needle aspirations. The use of these latter techniques is more common in early detection. Molecular and phenotypic analysis of cancer cells within a tissue can usually be used to confirm if the cancer is endogenous to the tissue or if the lesion is due to metastasis from another site. Some tumors are unresectable (cannot be surgically removed due to, for example the number of metastatic foci or because it is in a surgical danger zone). The methods of the invention can be utilized for early, middle, or late stage disease, and acute or chronic disease. The methods of the invention can be utilized for metastatic or non-metastatic cancer.

As used herein, the term “administration” is intended to include, but is not limited to, the following delivery methods: topical, oral, parenteral, subcutaneous, transdermal, transbuccal, intravascular (e.g., intravenous or intra-arterial), intramuscular, subcutaneous, intranasal, and intra-ocular administration. Administration can be local at a particular anatomical site, such as a site of a tumor, or systemic.

As used herein, the term “contacting” in the context of contacting a cell with at least one inhibitor or combination of inhibitors in vitro or in vivo means bringing at least one inhibitor into contact with the cell, or vice-versa, or any other manner of causing the inhibitor and the cell to come into contact.

The compounds of the present invention can be formulated into pharmaceutically-acceptable salt forms. Pharmaceutically-acceptable salts of the compounds of the invention can be prepared using conventional techniques. “Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, a “derivative” or “pharmaceutically active derivative” refers to any compound that upon administration to the recipient, is capable of providing directly or indirectly, the activity disclosed herein (e.g., inhibitory activity). The term “indirectly” also encompasses “prodrugs” which may be converted to the active form of the drug, e.g., via endogenous enzymes or metabolism (biotransformation). The prodrug is a derivative of the compounds according to the invention and presenting inhibitory activity that has a chemically or metabolically decomposable group, and a compound that may be converted into a pharmaceutically active compound according to the invention in vivo by solvolysis under physiological conditions. The prodrug is converted into a compound according to the present invention by a reaction with an enzyme, gastric acid or the like under a physiological condition in the living body, e.g., by oxidation, reduction, hydrolysis or the like, each of which is carried out enzymatically. These compounds can be produced from compounds of the present invention according to well-known methods. The term “indirectly” also encompasses metabolites of compounds according to the invention. Chemical reactions, reactants, and reagents useful for making derivatives can be found, for example, in March's Advanced Organic Chemistry, 7^{th} edition, 2013, Michael B. Smith, which is incorporated herein by reference in its entirety.

More specifically, the term “prodrug” refers to a chemical compound that can be converted by the body (i.e., biotransformed) to another chemical compound that has pharmacological activity. The prodrug may itself have pharmacological activity before conversion, or be inactive before conversion and activated upon conversion. Active prodrugs or inactive prodrugs of compounds of the invention may be administered to a subject or contacted with a cell in vitro or in vivo. Instead of administering a drug directly, a prodrug may be used instead to improve how a drug is absorbed, distributed, metabolized, and excreted (ADME). For example, a prodrug may be used to improve bioavailability when a drug itself is poorly absorbed from the gastrointestinal tract, or to improve how selectively the drug interacts with cells or processes that are not its intended target, which can reduce adverse or unintended effects of a drug. Major types of prodrugs include, but are not limited to, type I prodrugs, which are biotransformed inside cells (intracellularly), and type II prodrugs, which are biotransformed outside cells (extracellularly), such as in digestive fluids or in the body's circulatory system. These types can be further categorized into subtypes based on factors such as whether the intracellular bioactivation location is also a site of therapeutic action, or whether or not bioactivation occurs in the gastrointestinal fluids or in the circulation system (Wu, Kuei-Meng, “A New Classification of Prodrugs: Regulatory Perspectives, Pharmaceuticals, 2009, 2(3):77-81, which is incorporated by reference herein in its entirety).

The term “metabolite” refers to all molecules derived from any of the inhibitors according to the present invention in a cell or organism, preferably mammal. Pharmaceutically active metabolites of the compounds of the invention may be administered to a subject or contacted with a cell in vitro or in vivo.

The phrase “pharmaceutically acceptable” indicates that the substance or composition must be compatible chemically and/or toxicologically, with the other ingredients comprising a formulation, and/or the mammal being treated therewith.

Pharmaceutical formulations include “pharmaceutically acceptable” and “physiologically acceptable” carriers, diluents or excipients. In this context, the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the compositions.

The phrase “effective amount” means an amount of an agent, such as an inhibitor, that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, a subject is “in need of” a treatment if such human or non-human animal subject would benefit biologically, medically or in quality of life from such treatment (preferably, a human).

As used herein, the term “inhibit”, “inhibition” or “inhibiting” refers to the reduction or suppression of a given condition, symptom, or disorder, or disease (e.g., cancer), or a significant decrease in the baseline activity of a biological activity or process targeted by an inhibitor.

An “inhibitor of the invention” refers to the inhibitors of (a), (b), (c), (d), and (e), i.e., inhibitor of DNA-Dependent Protein Kinase catalytic subunit (DNA-PKcs inhibitor), Poly-ADP Ribose Polymerase (PARP inhibitor), inhibitor of wild-type isocitrate dehydrogenase (IDH inhibitor), inhibitor of histone acetyltransferase (HAT inhibitor), inhibitor of histone deacetylase (HDAC inhibitor), or any combination of two or more of the foregoing. In some embodiments, a single agent (e.g., a single compound) may have properties of two or more of the aforementioned classes of inhibitors, or have properties of further classes of inhibitors. For example, CC-115 is a dual DNA-PKcs inhibitor and an mTOR inhibitor.

The inhibitor may be any agent capable of reducing or disrupting the target molecule or signaling function of the target molecule. Exemplary classes of inhibitors include, but are not limited to, small molecules, and macromolecules or biologics such as antibodies (monoclonal or polyclonal antibodies, or antigen-binding fragments thereof), inhibitory polynucleotides or oligonucleotides that reduce target molecule transcription or translation (e.g., antisense, siRNA, shRNA). Therefore, “inhibition” or “inhibiting” encompasses both pharmacological blocking of the target and genetic deletion of or interference with target molecule coding sequences.

The terms “compounds of the present invention” or “agents of the invention” (unless specifically identified otherwise) refer to inhibitors of the invention, including salts thereof, as well as all stereoisomers (including diastereoisomers and enantiomers), rotamers, tautomers and isotopically labeled compounds (including deuterium substitutions), as well as inherently formed moieties (e.g., polymorphs, solvates and/or hydrates). For purposes of this invention, solvates and hydrates are generally considered compositions.

The term “a,” “an,” “the” and similar terms used in the context of the present invention (especially in the context of the claims) are to be construed to cover both the singular and plural unless otherwise indicated herein or clearly contradicted by the context. For example, the term “cell” includes a singular cell and a plurality of cells unless specified to the contrary; and the term “inhibitor” includes a singular inhibitor and a plurality of inhibitors.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about”.

### Materials and Methods

Cell lines, stable shRNA mediated H3.3 knockdown and plasmids. Human colon carcinoma cell line HCT116 and its derivatives were used in all the studies except where indicated. These cells were routinely cultured at 37° C. and 5% CO₂ in McCoy's 5A media with 10% fetal bovine serum and 1× antibiotic/antimycotic cocktail (Invitrogen). Transfections were carried out using 1 μg of DNA corresponding to each expression construct with Lipofectamine 2000 (Invitrogen) following the manufacturer's protocol. Transiently transfected cells were assayed between 24 and 48 hours following transfection. Where needed, stable cell lines were generated by applying the selection antibiotic (1 g/ml G418) 48 hours following transfection and maintaining it for at least ˜3 weeks until individual colonies could be picked. Plasmids for the expression of canonical H3-GFP, H4-GFP (Dinant et al., 2007, 2008) and RFP-PCNA (Essers et al., 2005; Haince et al., 2008) have been described previously. Stable H3.3 knockdown was generated by employing lentiviral mediated simultaneous transduction of HCT116 cells with 6 different Mission shRNA constructs (Sigma-Aldrich) targeting both H3F3A and H3F3B genes followed by selection with 5 μg/ml puromycin for ˜2 weeks to obtain resistant clones carrying the H3.3 knockdown. The FLAG-tagged mouse H3.3 expression construct was a kind gift from Dr. Mitsuru Okuwaki and this construct is naturally resistant to the shRNA constructs used to knockdown H3.3 in human cells due to substantial differences in the nucleotide sequence despite identical H3.3 protein sequences between mice and humans. To construct the H3.3-mEmerald expression construct, a PCR amplified mouse H3.3 gene fused at its C-terminus to mEmerald (a brighter variant of eGFP) was ligated between the unique BglII and NotI restriction sites in a modified vector derived from pEGFP-C1 (Clontech).

Survival assays. To determine the sensitivity of cells to NHEJ inhibitors, IDH inhibitors, HATi or HDACi in combination with ionizing radiation, 25,000 cells were seeded per well in triplicate in 12-well cell culture plates in the presence of the indicated NHEJ inhibitors. Following reattachment of the cells to the bottom of the culture plate, they were irradiated with the indicated doses of X-rays using an X-Rad 320 irradiator (Precision X-Ray). Accurate X-ray dose delivery was determined using UNIDOS E dosimeter. Surviving cells were measured 7 days post-irradiation by first dissociating the cells with trypsin and then counting them on a Z3 Coulter counter (Beckman-Coulter). Experiments were typically repeated at least 3 times.

Western Blotting and Immunofluorescence (IF). Our detailed protocol for Western blotting as well as the histone H3 and H4 antibodies used have been described previously (Kimura and Cook, 2001; Polo et al., 2006). IF was carried out using standard procedures. Home-made affinity purified rabbit polyclonal antibodies for γH2A.X were used with identical results in Western Blotting and IF as commercially available mouse monoclonal γH2A.X antibodies (Millipore). Additionally, the following commercially available antibodies were used for Western blotting and/or IF: and H3.3 (Millipore), H3.3 (Abnova), GFP and RFP (Rockland Immunochemicals).

Live cell imaging following laser microirradiation induced DNA damage. Fluorescently (mEmerald) tagged mouse wild type or mutant mouse H3.3 (which is identical to the human H3.3 protein) were transiently or stably expressed in HCT116 human colon carcinoma cells to study their recruitment to sites of laser microirradiation induced DNA damage by live cell imaging. Importantly, no chemical treatments with nucleotide analogs such as BrdU, or DNA intercalating dyes such as Hoechst or DAPI to pre-sensitize the cells to DNA damage was used, since these treatments could potentially interfere with our experiments and give rise to unanticipated effects due to their potential for modulating chromatin structure upon incorporation into DNA. For laser induced DNA damage and imaging experiments, cells were grown and transfected in collagen coated glass bottom 35 mm culture dishes (Mattek) inside a humidified chamber maintained at 5% CO₂ and 37° C. on the stage of a Andor Revolution spinning disk live cell imaging system equipped with a Nikon Eclipse TiE inverted confocal microscope with the Perfect Focus system. DNA damage was typically inflicted in a ˜0.25 μm diffraction limited spot (or a ˜0.25 μm thick line) within the nucleus using the Andor FRAPPA system to fire a 100 mW 405 nm laser at 100% power for 1 ms on a diffraction limited spot through 60× or 100× Nikon 1.49NA TIRF objectives. This was followed by rapid imaging (at least 1 frame per channel per second for the first 2 minutes and every 10-30s thereafter) using an Andor iXON 897 EMCCD camera to follow any recruitment of the tagged proteins to the site of laser induced DNA damage. The rapid recruitment of fluorescently (Red Fluorescent Protein, RFP) tagged DNA replication factors such as PCNA (Doil et al., 2009; Haince et al., 2008) and chromatin assembly factors such as CAF1 (Zeitlin et al., 2009) that are known to be recruited to sites of DNA damage was used as positive controls in these experiments.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Histones Bind to IDH Enzymes and Increase their Catalytic Activity

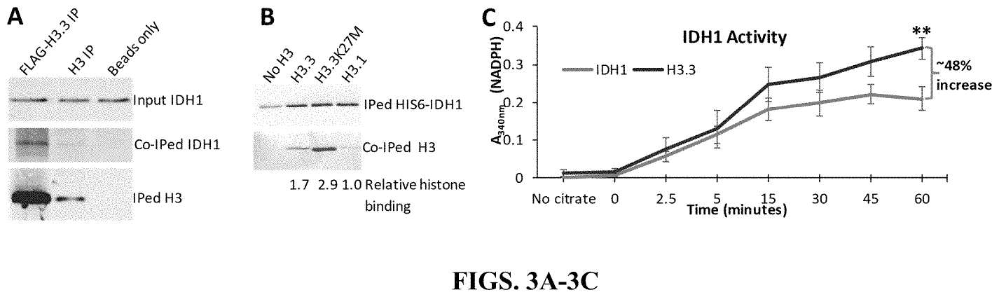

While analyzing proteins associated with histone H3.3 immunoprecipitated (IPed) from human whole cell extracts (WCEs) by mass spectrometry (MS), we discovered that histone H3 variants were reproducibly associated with IDH enzymes in six independent experiments (data not shown). This was surprising because histones are primarily localized in the nucleus whereas IDH1 is cytosolic and IDH2/IDH3 are localized in the mitochondria. Nevertheless, all proteins are synthesized in the cytoplasm where they have the opportunity to interact, prior to heading to their final destinations within different cellular compartments. In fact, the presence of small amounts of histones in the cytoplasm has been documented before (Cook et al., 2011; Singh et al., 2012) and since histones are very abundant proteins, this small cytoplasmic pool can exert significant physiological effects. Conversely, the regulation of histone genes by other metabolic enzymes such as GAPDH has also been reported previously (Zheng et al., 2003). We confirmed our MS results by reciprocal analysis of the proteins associated with human H3 using immunoprecipitation (IP) experiments, which clearly demonstrated the presence of the IDH enzymes in association with histone H3 (FIG. 3A). IDH1 appeared to be preferentially associated with the H3.3 variant over canonical H3. Next, using in vitro binding and IDH1 activity assays with purified recombinant human proteins, we observed that H3.3 binds directly to IDH1, with the H3.3 K27M mutant exhibiting a higher affinity for binding to IDH1 than wild type H3.3 or H3.1 (FIG. 3B), consistent with our findings in FIG. 3A. Further, inclusion of H3.3 in the IDH1 reaction significantly enhanced the enzymatic activity of IDH1 in vitro (FIG. 3C). Taken together, our in vitro data suggests that cells carrying the H3.3 K27M mutant are likely to exhibit higher IDH1 activity and elevated α-KG levels, which in turn would lead to excessive demethylation reactions, consistent with the DNA and histone hypomethylation observed in H3.3 K27M mutant cells (Bender et al., 2013; Venneti et al., 2013).

### Example 2-H3.3 K27M Cells Exhibit Low Levels of Histone and DNA Methylation which can be Reversed Using Inhibitors of Wild Type IDH1

We directly tested the SF7761 pediatric DIPG cells carrying the H3.3 K27M mutant and confirmed that they do indeed exhibit very low levels of H3 K27me3 (FIG. 4A; Chan et al., 2013; Lewis et al., 2013) as well as DNA methylation (FIG. 4B; Bender et al., 2013). The low levels of H3 K27Me3 are not simply due to the H3.3 K27M mutation as the mutant comprises of only about 5-15% of the total H3 in these cells, with the majority of the H3 in these cells being wild type. Hence, a low level of H3.3 K27M mutant histone is believed to act in a dominant negative manner by sequestering the PRC2 complex that methylates H3 K27, thereby giving rise to the low levels of H3 K27me3 observed in the mutant DIPG cells (Chan et al., 2013; Lewis et al., 2013), although recent reports suggest that this mechanism is likely to be more complicated (Stafford et al., 2018; Yu et al., 2019). Next, we tested if inhibiting IDH1 in these H3.3K27M mutant cells could reverse the DNA and histone hypomethylation in H3.3 K27M mutant cells. Exposing SF7761 cells carrying the H3.3K27M mutation to an IDH1 inhibitor led to an increase in both H3K27me3 levels (FIG. 4C) and DNA methylation levels (FIG. 4D). No significant effect of the IDH1 inhibitor was observed on the H3K27me3 levels or the DNA methylation levels in cells carrying wild type H3.3 (data not shown).

Next, we tested if inhibiting IDH1 in these H3.3K27M mutant cells could reverse the DNA and histone hypomethylation in H3.3 K27M mutant cells. Exposing SF7761 cells carrying the H3.3K27M mutation to an IDH1 inhibitor led to an increase in both H3K27me3 levels (FIG. 4C) and DNA methylation levels (FIG. 4D). No significant effect of the IDH1 inhibitor was observed on the H3K27me3 levels or the DNA methylation levels in cells carrying wild type H3.3 (data not shown).

### Example 3—Patient-Derived H3.3 K27M Mutant Cancer Cells are Sensitive to Inhibitors of Wild Type IDH1

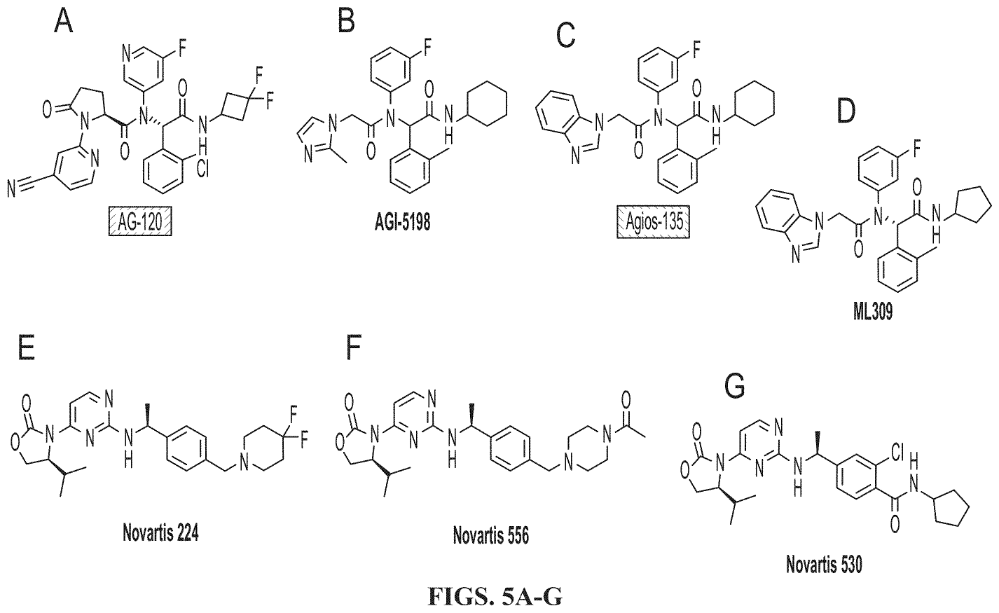

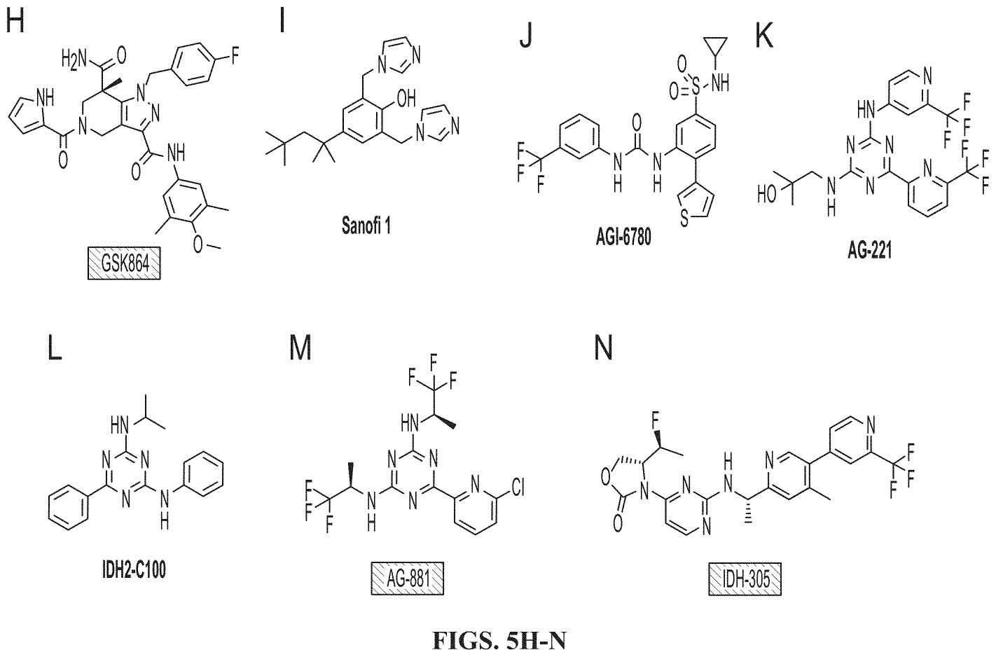

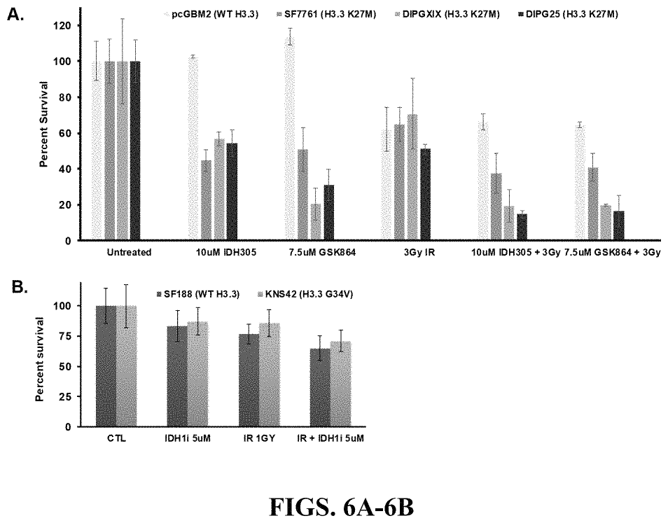

Next, we tested several H3.3 K27M mutant DIPG cells for sensitivity to different commercially available IDH1 inhibitors that inhibit mutant IDH1 in nano molar range, but also inhibit wild type IDH1 at higher concentrations (FIGS. 5A-5N). The H3.3 K27M mutant tumor cells were very sensitive to several IDH1 inhibitors (FIG. 6A). Remarkably, IDH1 inhibition appeared to show a synergistic effect with DNA damage caused by ionizing radiation (IR) treatment in eliminating several patient derived H3.3 K27M mutant DIPG cells (FIG. 6A). This synergy between IDH1 inhibition and radiation is not surprising at all given that DNA methylation levels are known to impact the radiation sensitivity of cells and this has been getting increasing attention in recent years as a possible target for improving the efficiency of radiation therapy for cancer (Chi et al., 2018; Ou et al., 2018; Zielske, 2015; Zhu et al., 2018). Additionally, histone modifications including methylation have also implicated in modulating the efficiency of radiation therapy (Gursoy-Yuzugullu et al., 2017; Smits et al., 2014) and a histone H3 demethylase inhibitor has been reported to enhance the efficacy of radiotherapy of DIPG tumors xenografted in mice (Katagi et al., 2019). Furthermore, histones accumulate transiently upon DNA damage (Gunjan and Verreault, 2003) and the interaction between histones and IDH proteins is also enhanced upon DNA damage (data not shown). It is possible that the regulation of IDH enzymes by histones and their downstream effects on histone and DNA methylation may be physiologically important for survival following DNA damage. Importantly, the inhibitory effect of the IDH1 inhibitors on cell growth was very selective for the H3.3 K27M mutant DIPG cells since other patient derived pediatric glioblastoma cells carrying either wild type H3.3 (SF188 cells) or the H3.3 G34V mutation (KNS42 cells) (Bjerke et al., 2013) were not significantly sensitive to IDH1 inhibition (FIG. 6B). This data strongly suggests that despite IDH1 being an important metabolic enzyme, any therapeutic strategy using IDH1 inhibitors is likely to spare the majority of normal non-tumor cells carrying wild type H3.3 and will selectively eliminate H3.3 K27M mutant tumor cells.

Currently, no effective therapies are available for high grade pediatric glioblastomas, and patients receive mostly palliative care (MacDonald et al., 2011). Hence, these patients are in a desperate need of effective therapies. Recent preclinical studies have studied the effectiveness of a histone demethylase and deacetylase inhibitors to ameliorate the low H3 K27me3 levels in H3.3 K27M mutant cancers (Grasso et al., 2015; Hashizume et al., 2014). However, these inhibitors only enhance the low H3 K27me3 levels in the H3.3 K27M mutant cancer cells but not their DNA hypomethylation. The α-KG dependent TET2 enzyme has recently been reported to alter gene expression by demethylating enhancers (Wang et al., 2018), highlighting potential importance of reversing both histone and DNA hypomethylation in H3.3 K27M mutant DIPG cells for maximal therapeutic benefit. Therefore, our strategy is all the more attractive because it involves the use of IDH1 inhibitors that can downregulate α-KG levels and thereby increase both histone and DNA methylation levels in the H3.3 K27M mutant cancer cells, thus sensitizing them to standard radiotherapy.

Overall, our data suggest that H3.3 and IDH proteins interact physically and functionally. Further, our data suggest that mutations in either protein may result in dysregulation of similar molecular pathways, thereby resulting in glioblastomas in both adults and children possibly via similar mechanisms. Most importantly, our results suggest novel therapeutic options involving the inhibition of wild type IDH enzymes to treat currently incurable H3.3 K27M mutant pediatric tumors. Finally, based on our research findings with H3.3 K27M mutant DIPG tumors, we suggest that inhibitors for wild type IDH enzymes may also be useful for treating additional tumors, especially those exhibiting histone and/or DNA hypomethylation, or overexpressing IDH enzymes.

### Example 4—Cells Deficient in H3.3 are Impaired in the Recruitment of HR Factors to Sites of DNA Damage and Exhibit Elevated Rates of NHEJ

Compelling preliminary data from three different eukaryotic species (only human data presented here) show that histone H3.3 plays an important role in surviving a variety of DNA damaging agents, including alkylating agents such as Methyl Methane Sulfonate (MMS) and strand break agents such as IR and Bleocin. Further, a recent report using chicken cells lacking H3.3 also suggests that H3.3 is involved in regulating replication fork progression following UV damage (Frey et al., 2014). Since these genotoxic agents cause very different types of DNA lesions that are repaired by distinct repair pathways, our data suggest that H3.3 is either involved in facilitating repair via more than one pathway, or that it functions at a step that is common to multiple DNA repair pathways, such as generating chromatin permissive for repair. To test which DNA repair pathways are affected by H3.3 deficiency, we measured the recruitment of repair factors specific to different DNA repair pathways using our live cell imaging-based assay to follow repair at sites of laser microirradiation that results in multiple types of DNA damage (Dinant et al., 2007). We did not find any differences between control and H3.3 KD cells in the recruitment efficiency of repair factors involved in Base Excision Repair (BER), Nucleotide Excision Repair (NER) or Mis-Match Repair (MMR). On the other hand, the recruitment of HR factor PALB2 (Partner and Localizer of BRCA2) was drastically impaired in H3.3 KD cells (FIGS. 10A, 10B), while the recruitment of BRCA1 and RPA was modestly reduced (data not shown), suggesting a specific defect in HR in the H3.3 KD cells. Importantly, the recruitment of RPA was ˜5-fold slower in H3.3 deficient cells (FIG. 10C), suggesting that DSB resection required to generate ssDNA for RPA binding may be slow or defective in H3.3 KD cells, which would be consistent with a role for H3.3 in generating accessible chromatin at DNA damage sites to facilitate repair. A role for H3.3 in HR mediated repair is also suggested by the sensitivity of H3.3KD cells to IR only upon release of serum starved cells from G1 arrest into S-phase, but not when they are arrested in G1 where NHEJ is the predominant repair pathway (FIG. 10D). Further, direct measurement of the HR mediated repair of an I-SceI cleavage site within the GFP gene as described previously (Fung and Weinstock, 2011) revealed a strong HR defect in H3.3 KD cells (FIG. 10E).

Next, we measured the efficiency of NHEJ in our HCT116 derived H3.3 KD cells and found that they exhibited double the levels of NHEJ compared to LUC cells (FIG. 10F), perhaps to compensate for their HR defect. Consistent with this observation, the recruitment of KU80-GFP was modestly faster in H3.3 KD cells (data not shown). Additionally, the kinetics of recruitment for the DSB pathway choice factor p53BP1 (p53 Binding Protein 1, which inhibits resection to suppress HR, thereby promoting NHEJ; Panier & Boulton, 2014), was significantly faster in the H3.3 KD cells (4.64 minutes in H3.3 KD cells compared to 6.13 minutes in control LUC cells; n=40; p=0.0002). We also created stable knockdown of H3.3 in U2OS cells carrying an I-SceI endonuclease and RFP based NHEJ reporter system (Bindra et al., 2013) and once again found double the levels of mutagenic-NHEJ mediated repair in H3.3 KD cells compared to controls even in U2OS cells (FIG. 10G). Finally, to further confirm the observed defect in HR and excessive reliance on NHEJ for survival following endogenous damage in H3.3 deficient cells, we treated the cells with NHEJ inhibitors and found them to be very sensitive compared to WT cells (FIG. 10H). These results are fully consistent with a specific defect in HR, but not NHEJ in the H3.3 deficient cells. Overall, our data show that H3.3 deficient cells are primarily defective in HR-mediated DSB repair rather than multiple DNA repair pathways and exhibit upregulated NHEJ. However, multiple types of DNA lesions can be processed during DNA replication or by different repair machineries to ultimately generate DSBs (McKinnon & Caldecott, 2007), it is likely that these secondary DSBs contribute to the observed sensitivity of H3.3 deficient cells to a variety of DNA damaging agents, rather than to DSB reagents alone.

### Example 5—Cells Carrying Cancer-Associated H3.3 Mutants are Sensitive to NHEJ Inhibitors

Our data suggest that H3.3 knockdown cells and those carrying cancer associated H3.3 mutations are likely to be defective in HR mediated repair and should be sensitive to NHEJ inhibitors due to synthetic lethality induced by the loss of all the DSB repair pathways, whereas normal cells with wild type H3.3 would be spared as they have a functional HR mediated DSB repair pathway. We tested this directly in our H3.3 knockdown cells using PARP1 inhibitor Olaparib to inhibit the A-EJ pathway and NU7441 to inhibit DNA-PKcs mediated c-NHEJ (Zhao et al., 2006; FIG. 7) and found them to be sensitive to both the drugs individually, especially when combined with IR treatment (FIG. 11A). Next, we confirmed these findings in the KNS42 pediatric glioblastoma cell line carrying the H3.3 G34V mutation which was also very sensitive to a combination of both the drugs and IR (FIG. 11B). Sensitivity to Olaparib has been previously reported for KNS42 cells (van Vuurden et al., 2011) and our data suggests that this is likely to be due to a defect in HR. However, isogenic WT cells are not available for the KNS42 cells for us to compare their sensitivities directly. Hence, we tested the sensitivity of our reconstituted isogenic WT and H3.3 G34R mutant cells to this combination of NHEJ inhibition and IR and obtained nearly identical results (FIG. 11C) to those obtained with KNS42 cells.

We have also tested the sensitivity of patient-derived Diffuse Intrinsic Pontine Glioma (DIPG) tumor cells carrying the H3.3 K27M mutation that are currently incurable and lack any approved therapies. Our results demonstrate that treatment with NHEJ inhibitors in combination with radiation mediated DNA strand breaks are also efficient in specifically eliminating patient derived H3.3 K27M mutant glioblastoma cells (SF7761 and SU-DIPG XIII both carrying the H3.3 K27M mutation), while cells with wild type H3.3 (PCGBM2) are spared (FIG. 11D).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Amary, M. F., Bacsi, K., Maggiani, F., Damato, S., Halai, D., Berisha, F., Pollock, R., O'Donnell, P., Grigoriadis, A., Diss, T., et al. (2011). IDH1 and IDH2 mutations are frequent events in central chondrosarcoma and central and periosteal chondromas but not in other mesenchymal tumours. The Journal of Pathology 224: 334-343.
Arimura, Y., Kimura, H., Oda, T., Sato, K., Osakabe, A., Tachiwana, H., Sato, Y., Kinugasa, Y., Ikura, T., Sugiyama, M., et al. (2013). Structural basis of a nucleosome containing histone H2A. B/H2A.Bbd that transiently associates with reorganized chromatin. Scientific Reports 3: 3510.
Barber, L. J., Sandhu, S., Chen, L., Campbell, J., Kozarewa, I., Fenwick, K., Assiotis, I., Rodrigues, D. N., Reis Filho, J. S., Moreno, V., et al. (2013). Secondary mutations in BRCA2 associated with clinical resistance to a PARP inhibitor. The Journal of Pathology 229: 422-429.
Behjati, S., Tarpey, P. S., Presneau, N., Scheipl, S., Pillay, N., Van Loo, P., Wedge, D. C., Cooke, S. L., Gundem, G., Davies, H., et al. (2013). Distinct H3F3A and H3F3B driver mutations define chondroblastoma and giant cell tumor of bone. Nature Genetics 45: 1479-1482.
Bender, S., Tang, Y., Lindroth, A. M., Hovestadt, V., Jones, D. T., Kool, M., Zapatka, M., Northcott, P. A., Sturm, D., Wang, W., et aL (2013). Reduced H3K27me3 and DNA hypomethylation are major drivers of gene expression in K27M mutant pediatric high-grade gliomas. Cancer Cell 24: 660-672.
Bennett, C. B., Westmoreland, T. J., Snipe, J. R., and Resnick, M. A. (1996). A double-strand break within a yeast artificial chromosome (YAC) containing human DNA can result in YAC loss, deletion or cell lethality. Mol. Cell. Biol. 16: 4414-4425.
Bjerke, L., Mackay, A., Nandhabalan, M., Burford, A., Jury, A., Popov, S., Bax, D. A., Carvalho, D., Taylor, KR., Vinci, M., et al. (2013). Histone H3.3 Mutations Drive Pediatric Glioblastoma through Upregulation of MYCN. Cancer Discovery 3: 512-519.
Brown, D. T. (2003). Histone H1 and the dynamic regulation of chromatin function. Biochem. Cell Biol. 81: 221-227.
Cairns, R. A., and Mak, T. W. (2013). Oncogenic isocitrate dehydrogenase mutations: mechanisms, models, and clinical opportunities. Cancer Discovery 3: 730-741.
Chan, K. M., Fang, D., Gan, H., Hashizume, R., Yu, C., Schroeder, M., Gupta, N., Mueller, S., James, C. D., Jenkins, R., et al. (2013). The histone H3.3K27M mutation in pediatric glioma reprograms H3K27 methylation and gene expression. Genes & Development 27: 985-990.
Chi, H. C., Tsai, C. Y., Tsai, M. M., and Lin, K. H. (2018). Impact of DNA and RNA Methylation on Radiobiology and Cancer Progression. Int J Mol Sci, 19: 555.
Ciccia, A., and Elledge, S. J. (2010). The DNA damage response: making it safe to play with knives. Molecular Cell 40: 179-204.
Cook, A. J., Gurard-Levin, Z. A., Vassias, I., and Almouzni, G. (2011). A specific function for the histone chaperone NASP to fine-tune a reservoir of soluble H3-H4 in the histone supply chain. Molecular Cell 44: 918-927.
Dang, L., White, D. W., Gross, S., Bennett, B. D., Bittinger, M. A., Driggers, E. M., Fantin, V. R., Jang, H. G., Jin, S., Keenan, M. C., et aL (2009). Cancer-associated IDH1 mutations produce 2-hydroxyglutarate. Nature 462: 739-744.
Davis, M. I., Gross, S., Shen, M., Straley, K. S., Pragani, R., Lea, W. A., Popovici-Muller, J., DeLaBarre, B., Artin, E., Thorne, N., et al. (2014). Biochemical, cellular, and biophysical characterization of a potent inhibitor of mutant isocitrate dehydrogenase IDH1. The Journal of Biological Chemistry 289: 13717-13725.
Dinant, C., de Jager, M., Essers, J., van Cappellen, W. A., Kanaar, R., Houtsmuller, A. B., and Vermeulen, W. (2007). Activation of multiple DNA repair pathways by sub-nuclear damage induction methods. J. Cell Sci. 120: 2731-2740.
Dinant, C., Houtsmuller, A. B., and Vermeulen, W. (2008). Chromatin structure and DNA damage repair. Epigenetics & Chromatin 1:9.
Doil, C., Mailand, N., Bekker-Jensen, S., Menard, P., Larsen, D. H., Pepperkok, R., Ellenberg, J., Panier, S., Durocher, D., Bartek, J., Lukas, J., and Lukas, C. (2009). RNF168 binds and amplifies ubiquitin conjugates on damaged chromosomes to allow accumulation of repair proteins. Cell 136:435-46.
Essers, J., Theil, A. F., Baldeyron, C., van Cappellen, W. A., Houtsmuller, A. B., Kanaar, R., and Vermeulen, W. (2005). Nuclear dynamics of PCNA in DNA replication and repair. Mol. Cell. Biol. 25: 9350-9359.
Fontebasso, A. M., Liu, X. Y., Sturm, D., and Jabado, N. (2013). Chromatin remodeling defects in pediatric and young adult glioblastoma: a tale of a variant histone 3 tail. Brain Pathology 23: 210-216.
Frey, A., Listovsky, T., Guilbaud, G., Sarkies, P., and Sale, J. E. (2014). Histone H3.3 Is Required to Maintain Replication Fork Progression after UV Damage. Current Biology 24: 2195-2201.
Fung, H., and Weinstock, D. M. (2011). Repair at single targeted DNA double-strand breaks in pluripotent and differentiated human cells. PloS one 6: e20514.
Gong, L., Wang, E., and Lin, S.-Y. (2013). “Chromatin Remodeling in DNA Damage Response and Human Aging” in Chromatin Remodelling, Dr. Danuta Radzioch (Ed.)
Grasso, C. S., Tang, Y., Truffaux, N., Berlow, N. E., Liu, L., Debily, M. A., Quist, M. J., Davis, L. E., Huang, E. C., Woo, P. J., et al. (2015). Functionally defined therapeutic targets in diffuse intrinsic pontine glioma. Nature Medicine 21: 555-559.
Gunjan, A., and Verreault, A. (2003). A Rad53 kinase-dependent surveillance mechanism that regulates histone protein levels in Saccharomyces cerevisiae. Cell 115: 537-549.
Gursoy-Yuzugullu, O., Carman, C., Serafim, R. B., Myronakis, M., Valente, V., and Price, B. D. (2017). Epigenetic therapy with inhibitors of histone methylation suppresses DNA damage signaling and increases glioma cell radiosensitivity. Oncotarget 8: 24518-24532.
Haince, J. F., McDonald, D., Rodrigue, A., Wry, U., Masson, J. Y., Hendzel, M. J., and Poirier, G. G. (2008). PARP1-dependent kinetics of recruitment of MRE11 and NBS1 proteins to multiple DNA damage sites. J. Biol. Chem. 283:1197-208.
Harper, J. W., and Elledge, S. J. (2007). The DNA damage response: ten years after. Molecular Cell 28: 739-745.
Hashizume, R., Andor, N., Ihara, Y., Lerner, R., Gan, H., Chen, X., Fang, D., Huang, X., Tom, M. W., Ngo, V., et al. (2014). Pharmacologic inhibition of histone demethylation as a therapy for pediatric brainstem glioma. Nature Medicine 20: 1394-1396.
Henikoff, S., Henikoff, J. G., Sakai, A., Loeb, G. B., and Ahmad, K. (2009). Genome-wide profiling of salt fractions maps physical properties of chromatin. Genome Research 19: 460-469.
Jang, C. W., Shibata, Y., Starmer, J., Yee, D., and Magnuson, T. (2015). Histone H3.3 maintains genome integrity during mammalian development. Genes & Development 29: 1377-1392.
Jaspers, J. E., Kersbergen, A., Boon, U., Sol, W., van Deemter, L., Zander, S. A., Drost, R., Wientjens, E., Ji, J., Aly, A., et al. (2013). Loss of 53BP1 causes PARP inhibitor resistance in Brcal-mutated mouse mammary tumors. Cancer Discovery 3: 68-81.
Jin, C., and Felsenfeld, G. (2007). Nucleosome stability mediated by histone variants H3.3 and H2A. Z. Genes & Development 21: 1519-1529.
Karagiannis, T. C., and El-Osta, A. (2006). Modulation of cellular radiation responses by histone deacetylase inhibitors. Oncogene 25: 3885-93.
Katagi, H., Louis, N., Unruh, D., Sasaki, T., He, X., Zhang, A., Ma, Q., Piunti, A., Shimazu, Y., Lamano, J. B., et al. (2019). Radiosensitization by Histone H3 Demethylase Inhibition in Diffuse Intrinsic Pontine Glioma. Clin Cancer Res 25: 5572-5583.
Kimura, H., and Cook, P. R. (2001). Kinetics of core histones in living human cells: little exchange of H3 and H4 and some rapid exchange of H2B. J. Cell Biol. 153: 1341-1353.
Lewis, P. W., Muller, M. M., Koletsky, M. S., Cordero, F., Lin, S., Banaszynski, L. A., Garcia, B. A., Muir, T. W., Becher, O. J., and Allis, C. D. (2013). Inhibition of PRC2 activity by a gain-of-function H3 mutation found in pediatric glioblastoma. Science 340: 857-861.
Liu M. W., DeNizio, J. E., and Kohli, R. M. (2016). Quantification of Oxidized 5-Methylcystosine Bases and TET Enzyme Activity. Methods Enzymol, 573:365-385.
Losman, J. A., Looper, R. E., Koivunen, P., Lee, S., Schneider, R. K., McMahon, C., Cowley, G. S., Root, D. E., Ebert, B. L., and Kaelin, W. G., Jr. (2013). (R)-2-hydroxyglutarate is sufficient to promote leukemogenesis and its effects are reversible. Science 339: 1621-1625.
Lu, C., Ward, P. S., Kapoor, G. S., Rohle, D., Turcan, S., Abdel-Wahab, O., Edwards, C. R., Khanin, R., Figueroa, M. E., Melnick, A., et al. (2012). IDH mutation impairs histone demethylation and results in a block to cell differentiation. Nature 483: 474-478.
MacDonald, T. J., Aguilera, D., and Kramm, C. M. (2011). Treatment of high-grade glioma in children and adolescents. Neuro-oncology 13: 1049-1058.
Marzluff, W. F., Gongidi, P., Woods, K. R., Jin, J., and Maltais, L. J. (2002). The human and mouse replication-dependent histone genes. Genomics 80: 487-498.
McKinnon, P. J., and Caldecott, K. W. (2007). DNA strand break repair and human genetic disease. Annual Review of Genomics and Human Genetics 8: 37-55.
Nijman, S. M. (2011). Synthetic lethality: general principles, utility and detection using genetic screens in human cells. FEBS Letters 585: 1-6.
Ou, Y., Zhang, Q., Tang, Y., Lu, Z., Lu, X., Zhou, X., and Liu, C. (2018). DNA methylation enzyme inhibitor RG108 suppresses the radioresistance of esophageal cancer. Oncol Rep 39: 993-1002.
Panier, S., and Boulton, S. J. (2014). Double-strand break repair: 53BP1 comes into focus. Nature reviews Molecular Cell Biology 15: 7-18.
Polo, S. E., Roche, D., and Almouzni, G. (2006). New histone incorporation marks sites of UV repair in human cells. Cell 127: 481-493.
Richmond, T. J., and Davey, C. A. (2003). The structure of DNA in the nucleosome core. Nature 423: 145-150.
Rohle, D., Popovici-Muller, J., Palaskas, N., Turcan, S., Grommes, C., Campos, C., Tsoi, J., Clark, O., Oldrini, B., Komisopoulou, E., et al. (2013). An inhibitor of mutant IDH1 delays growth and promotes differentiation of glioma cells. Science 340: 626-630.
Sansoni, V., Casas-Delucchi, C. S., Rajan, M., Schmidt, A., Bonisch, C., Thomae, A. W., Staege, M. S., Hake, S. B., Cardoso, M. C., and Imhof, A. (2014). The histone variant H2A.Bbd is enriched at sites of DNA synthesis. Nucleic Acids Research 42: 6405-6420.
Schwartzentruber, J., Korshunov, A., Liu, X. Y., Jones, D. T., Pfaff, E., Jacob, K., Sturm, D., Fontebasso, A. M., Quang, D. A., Tonjes, M., et al. (2012). Driver mutations in histone H3.3 and chromatin remodelling genes in paediatric glioblastoma. Nature 482: 226-231.
Shih, A. H., and Levine, R. L. (2012). IDH1 mutations disrupt blood, brain, and barriers. Cancer Cell 22: 285-287.
Singh, R. K., Gonzalez, M., Kabbaj, M. H., and Gunjan, A. (2012). Novel E3 ubiquitin ligases that regulate histone protein levels in the budding yeast Saccharomyces cerevisiae. PloS One 7: e36295.
Smits, K. M., Melotte, V., Niessen, H. E., Dubois, L., Oberije, C., Troost, E. G., Starmans, M. H., Boutros, P. C., Vooijs, M., van Engeland, M., et al. (2014). Epigenetics in radiotherapy: where are we heading? Radiother Oncol 111: 168-177.
Stafford, J. M., Lee, C. H., Voigt, P., Descostes, N., Saldana-Meyer, R., Yu, J. R., Leroy, G., Oksuz, O., Chapman, J. R., Suarez, F., et al. (2018). Multiple modes of PRC2 inhibition elicit global chromatin alterations in H3K27M pediatric glioma. Sci Adv 4: eaau5935.
Suijker, J., Baelde, H. J., Roelofs, H., Cleton-Jansen, A. M., and Bovee, J. V. (2015). The oncometabolite D-2-hydroxyglutarate induced by mutant IDH1 or -2 blocks osteoblast differentiation in vitro and in vivo. Oncotarget 6: 14832-14842.
Szenker, E., Ray-Gallet, D., and Almouzni, G. (2011). The double face of the histone variant H3.3. Cell Research 21: 421-434.
Talbert, P. B., and Henikoff, S. (2010). Histone variants-ancient wrap artists of the epigenome. Nat Rev Mol. Cell Biol. 11: 264-275.
Tsukuda, T., Fleming, A. B., Nickoloff, J. A., and Osley, M. A. (2005). Chromatin remodelling at a DNA double-strand break site in Saccharomyces cerevisiae. Nature 438: 379-383.
Turcan, S., Rohle, D., Goenka, A., Walsh, L. A., Fang, F., Yilmaz, E., Campos, C., Fabius, A. W., Lu, C., Ward, P. S., et al. (2012). IDH1 mutation is sufficient to establish the glioma hypermethylator phenotype. Nature 483: 479-483.
Urban, D. J., Martinez, N. J., Davis, M. I., Brimacombe, K. R., Cheff, D. M., Lee, T. D., Henderson, M. J., Titus, S. A., Pragani, R., Rohde, J. M., et al. (2017). Assessing inhibitors of mutant isocitrate dehydrogenase using a suite of pre-clinical discovery assays. Sci Rep 7: 12758.
van Vuurden, D. G., Hulleman, E., Meijer, O. L., Wedekind, L. E., Kool, M., Witt, H., Vandertop, P. W., Wurdinger, T., Noske, D. P., Kaspers, G. J., et al. (2011). PARP inhibition sensitizes childhood high grade glioma, medulloblastoma and ependymoma to radiation. Oncotarget 2: 984-996.
Venneti, S., Garimella, M. T., Sullivan, L. M., Martinez, D., Huse, J. T., Heguy, A., Santi, M., Thompson, C. B., and Judkins, A. R. (2013). Evaluation of histone 3 lysine 27 trimethylation (H3K27me3) and enhancer of Zest 2 (EZH2) in pediatric glial and glioneuronal tumors shows decreased H3K27me3 in H3F3A K27M mutant glioblastomas. Brain Pathology 23: 558-564.
Wang, F., Travins, J., DeLaBarre, B., Penard-Lacronique, V., Schalm, S., Hansen, E., Straley, K., Kernytsky, A., Liu, W., Gliser, C., et al. (2013). Targeted inhibition of mutant IDH2 in leukemia cells induces cellular differentiation. Science 340: 622-626.
Wang, L., Ozark, P. A., Smith, E. R., Zhao, Z., Marshall, S. A., Rendleman, E. J., Piunti, A., Ryan, C., Whelan, A. L., Helmin, K. A., et aL (2018). TET2 coactivates gene expression through demethylation of enhancers. Sci Adv 4: eaau6986.
Wen, H., Li, Y., Xi, Y., Jiang, S., Stratton, S., Peng, D., Tanaka, K., Ren, Y., Xia, Z., Wu, J., et al. (2014). ZMYND11 links histone H3.3K36me3 to transcription elongation and tumour suppression. Nature 508: 263-268.
Willmore, E, de Caux, S., Sunter, N. J., Tilby, M. J., Jackson, G. H., Austin, C. A., and Durkacz, B. W. (2004). A novel DNA-dependent protein kinase inhibitor, NU7026, potentiates the cytotoxicity of topoisomerase II poisons used in the treatment of leukemia. Blood. 103:4659-65.
Wu, G., Broniscer, A., McEachron, T. A., Lu, C., Paugh, B. S., Becksfort, J., Qu, C., Ding, L., Huether, R., Parker, M., et al. (2012). Somatic histone H3 alterations in pediatric diffuse intrinsic pontine gliomas and non-brainstem glioblastomas. Nature Genetics 44: 251-253.
Xu, G., Chapman, J. R., Brandsma, I., Yuan, J., Mistrik, M., Bouwman, P., Bartkova, J., Gogola, E., Warmerdam, D., Barazas, M., et al. (2015). REV7 counteracts DNA double-strand break resection and affects PARP inhibition. Nature 521: 541-544.
Xu, Y., Ayrapetov, M. K., Xu, C., Gursoy-Yuzugullu, O., Hu, Y., and Price, B. D. (2012). Histone H2A. Z controls a critical chromatin remodeling step required for DNA double-strand break repair. Mol. Cell 48: 723-733.
Yan, H., Parsons, D. W., Jin, G., McLendon, R., Rasheed, B. A., Yuan, W., Kos, I., Batinic-Haberle, I., Jones, S., Riggins, G. J., et aL (2009). IDH1 and IDH2 mutations in gliomas. The New England Journal of Medicine 360: 765-773.
Yang, X., Li, L., Liang, J., Shi, L., Yang, J., Yi, X., Zhang, D., Han, X., Yu, N., and Shang, Y. (2013). Histone acetyltransferase 1 promotes homologous recombination in DNA repair by facilitating histone turnover. J. Biol. Chem. 288: 18271-18282.
Yu, J. R., Lee, C. H., Oksuz, O., Stafford, J. M., and Reinberg, D. (2019). PRC2 is high maintenance. Genes Dev 33: 903-935.
Zeitlin, S. G., Baker, N. M., Chapados, B. R., Soutoglou, E., Wang, J. Y., Berns, M. W., and Cleveland, D. W. (2009). Double-strand DNA breaks recruit the centromeric histone CENP-A. Proc. Natl. Acad. Sci. USA 106: 15762-15767.
Zhao, Q., Manning, J. R., Sutton, J., Costales, A., Sendzik, M., Shafer, C. M., Levell, J. R., Liu, G., Caferro, T., Cho, Y. S., et aL (2018). Optimization of 3-Pyrimidin-4-yl-oxazolidin-2-ones as Orally Bioavailable and Brain Penetrant Mutant IDH1 Inhibitors. ACS Med Chem Lett 9: 746-751.
Zhao, Y., Thomas, H. D., Batey, M. A., Cowell, I. G., Richardson, C. J., Griffin, R. J., Calvert, A. H., Newell, D. R., Smith, G. C., and Curtin, N. J. (2006). Preclinical evaluation of a potent novel DNA-dependent protein kinase inhibitor NU7441. Cancer Research 66: 5354-5362.
Zheng. L., Roeder, R. G., Luo, Y. (2003). S phase activation of the histone H2B promoter by OCA-S, a coactivator complex that contains GAPDH as a key component. Cell 114: 255-66.
Zhu, X., Wang, Y., Tan, L., and Fu, X. (2018). The pivotal role of DNA methylation in the radio-sensitivity of tumor radiotherapy. Cancer Med 7: 3812-3819.
Zielske, S. P. (2015). Epigenetic DNA methylation in radiation biology: on the field or on the sidelines? J Cell Biochem 116: 212-217.

I claim:

1. A method for treating or delaying the onset, progression, or relapse of a cancer in a human or non-human animal subject, the method selected from among:
(a) administering an inhibitor of DNA-Dependent Protein Kinase catalytic subunit (DNA-PKcs inhibitor), and an inhibitor of Poly-ADP Ribose Polymerase (PARP inhibitor) to the subject, wherein the cancer has a histone H3.3 mutation (such as K27M, G34R/V/W/L, or K36M mutation) or is a homologous recombination-defective (HR-defective) cancer; or
(b) administering an inhibitor of wild-type isocitrate dehydrogenase (IDH inhibitor) to the subject, wherein the IDH inhibitor is administered in an effective amount to decrease wild-type IDH activity in cells of the cancer, and wherein the cancer carries a histone H3.3 K27M mutation, and/or the cancer has low DNA methylation and/or low histone methylation; or
(c) administering an inhibitor of histone acetyltransferase (HAT inhibitor) to the subject, wherein the cancer carries a histone H3.3 K27M mutation, and/or has high histone acetylation; or
(d) administering an inhibitor of histone deacetylase (HDAC inhibitor) to the subject, wherein the cancer carries a histone H3.3 K27M mutation, and/or has high histone acetylation; or
(e) a combination of two or more of (a), (b), (c), or (d), wherein the inhibitors are administered to the subject simultaneously, or sequentially in any order.

2. The method of claim 1, wherein the method comprises (a), further comprising administering radiation therapy to the subject before, during, and/or after administering the DNA-PKcs inhibitor and PARP inhibitor.

3. The method of claim 1, wherein the method comprises (a), and wherein the cancer comprises cancer cells bearing an H3.3 mutation, resulting in defective double strand break (DSB) repair by the homologous recombination pathway.

4. The method of claim 1, wherein the method comprises (a), and wherein the cancer is glioblastoma comprising cancer cells bearing an H3.3 mutation.

5. The method of claim 1, wherein the method comprises (a), and wherein the cancer is breast cancer, ovarian cancer, pancreatic cancer, prostate cancer, or melanoma bearing one or more BRCA mutations (e.g., bearing BRCA1 and/or BRCA2 mutations).

6. The method of claim 1, wherein the method comprises (a), and wherein the cancer is any cancer exhibiting a defective homologous recombination pathway.

7. The method of claim 1 wherein the method comprises (a), and wherein the DNA-PKcs inhibitor comprises one or more compounds selected from among NU7441 (2-N-morpholino-8-dibenzothiophenyl-chromen-4-one, a.k.a. KU-57788), NU7026 (2-(morpholin-4-yl)-benzo[h]chromen-4-one), SU11752, NK314, AZD7648, M3814, VX-984, and CC-115.

8. The method of claim 1, wherein the method comprises (a), and wherein the PARP inhibitor comprises one or more compounds selected from among olaparib, rucaparib, niraparib, talzoparib, veliparib, BGB-290 (pamiparib), CEP 9722, E7016, and 3-aminobenzamide.

9. The method of claim 1, wherein the method comprises (b), and wherein the cancer is a glioblastoma or chondroblastoma.

10. The method of claim 1, wherein the method comprises (b), and wherein the cancer is a cancer exhibiting DNA hypomethylation or histone hypomethylation, or both.

11. The method of claim 1, wherein the method comprises (b), and wherein the IDH inhibitor comprises IDH305, GSK864, or a combination thereof.

12. The method of claim 1, wherein the method comprises (c), and wherein the HAT inhibitor is curcumin, garcinol, anacardic acid, C646 or CPTH2.

13. The method of claim 1, wherein the method comprises (c), and wherein the cancer is a cancer exhibiting histone hyperacetylation.

14. The method of claim 1, wherein the method comprises (d), and wherein the HDAC inhibitor comprises vorinostat, valproic acid, or a combination thereof.

15. The method of claim 1, wherein the method comprises (d), further comprising administering radiation therapy to the subject before, during, and/or after administering the HDAC inhibitor to the subject.

16. The method of claim 1, wherein the method comprises (b), and wherein the cancer is any cancer exhibiting histone hyperacetylation.

17. The method of claim 1, wherein the method comprises (a), (b), (c), (d), or (e), and wherein the cancer is diffuse intrinsic pontine glioma (DIPG).

18. The method of claim 1, wherein the method comprises (a), (b), (c), (d), or (e), further comprising administering radiation therapy to the subject before, during, and/or after administering the inhibitor.

19. A composition comprising an inhibitor of DNA-Dependent Protein Kinase catalytic subunit (DNA-PKcs inhibitor), and an inhibitor of Poly-ADP Ribose Polymerase (PARP inhibitor).

20. A composition comprising two or more of the following:
(a) an inhibitor of DNA-Dependent Protein Kinase catalytic subunit (DNA-PKcs inhibitor), and/or an inhibitor of Poly-ADP Ribose Polymerase (PARP inhibitor);
(b) a wild-type isocitrate dehydrogenase (IDH inhibitor);
(c) a histone acetyltransferase (HAT inhibitor); and
(d) histone deacetylase (HDAC inhibitor).
